(19) United States
(12) Patent Application Publication
(10) Pub. No.: US 2025/0288500 A1
(43) Pub. Date: Sep. 18, 2025

(54) An Improved Mineral Trioxide Aggregate Filling Material for Dentistry and a Device for its Use

(71) Applicant: PDS Inc., North Vancouver (CA)

(72) Inventors: Mahmoud TORABINEJAD, Laguna Niguel, CA (US); Mason A. BAHADOR, Baltimore, MD (US); Mehdi TALWERDI, North Vancouver, BC (CA); Winfield S. Fisher, Maple Ridge, BC (CA); Bahador SOHRABI, Vancouver, BC (CA)

(73) Assignee: PDS Inc., North Vancouver, BC (CA)

(21) Appl. No.: 18/860,381

(22) PCT Filed: Apr. 23, 2023
(86) PCT No.: PCT/CA2023/050550

§ 371 Date: Oct. 25, 2024

Related U.S. Application Data
(60) Provisional application No. 63/335,100, filed on Apr. 26, 2022.

(51) Int. Cl. A61K 6/851; A61C 5/50; A61C 5/64; A61C 5/66; A61C 5/68; A61K 6/73

(52) U.S. Cl. CPC A61K 6/851; A61C 5/50; A61C 5/64; A61C 5/66; A61C 5/68; A61K 6/73

(57) ABSTRACT

An improved Mineral Trioxide Aggregate (MTA) filling material for dentistry and a device for its use to seal natural, pathogenic, or iatrogenic, tooth cavities. The filling material comprises Portland cement, or variations in the composition of such cement, calcium sulfate and a radio pacifier material. The improved filling material exhibits favorable physical and chemical attributes sufficient to form a biocompatible and effective seal against re-entrance of infectious organisms. A device for delivery of MTA filling material to the site of operation is also provided.

### FIELD OF THE INVENTION

The present invention relates to an improved material and method of treating and filling cavities in teeth caused by decay or trauma for sealing the treated cavity or root canal to prevent infiltration of infectious bacteria into the living tissues of the tooth. In particular, the present invention relates to an improved Mineral Trioxide Aggregate (MTA) filling material and a device for its use to fill and/or seal tooth cavities.

### INTRODUCTION

The pulp tissue of mammalian teeth is encased in the root canal system, surrounded by dentin, and communicates with the periodontium through the apical foramen and, occasionally, small channels known as accessory or lateral canals. Removal of enamel and dentin by decay may result in communication between the root canal system, its dental pulp, and the periodontium. As a consequence of pulp exposure to the oral cavity, the root canal system acquires the ability to harbor bacteria and their byproducts. Because of its location, lack of enough collateral circulation, and its low compliance^{1,2}, the pulp does not have the ability to defend itself against the invading bacteria. Sooner or later the bacterial infection will spread throughout the root canal system and the bacteria and/or bacterial byproducts will diffuse from the root canal into the periradicular tissues with resultant development of a periradicular lesion. In addition, iatrogenic pathways of communication between the root canal system and the periodontium are created after accidental procedures such as perforations during access preparation, cleaning and shaping as well as post space preparation. In contrast to pulp tissue, periradicular tissues (periodontal ligament and bone) have an almost unlimited source of undifferentiated cells that can participate in the process of inflammation as well as repair. In addition, the periradicular tissues have rich collateral blood supply and lymph drainage. These characteristics enable the periradicular tissues to combat the destructive factors related to the irritants from the root canal system^{3,4}.

Numerous materials have been suggested to seal off the communication between the root canal system and external surfaces of the tooth. They include: gutta-percha, amalgam, polycarboxylate cements, zinc phosphate cements, zinc oxide eugenol paste, IRM cement, EBA cement, Cavit, glass ionomers, composite resins, and other materials such as gold foil and leaf, silver points, cyanoacrylates, polyHEMA and hydron, Diaket root canal sealer, titanium screws, and Teflon. For years, existing materials did not possess the “ideal” characteristics of a repair material, and therefore a material known as Mineral Trioxide Aggregate (MTA) was developed in 1993⁵. For example, U.S. Pat. Nos. 5,769,638 and 8,979,991 to Torabinejad et al., the contents of each are herein incorporated by reference, teach MTA for use in general dentistry and in endodontics to replace natural tooth material. MTA (sold under the trade names gray or white ProRoot® MTA (Dentsply International Inc., York, Pa., US) is a substance currently used in general dentistry and in endodontics and is used in apexification, pulp capping, pulpotomy, regenerative endodontics, root canal filling, root-end filling and root perforation repair⁶.

In a series of tests, Torabinejad and associates investigated in vitro dye leakage with and without blood contamination, in vitro bacterial leakage, scanning electron microscope (SEM) examination of replicas for marginal adaptation, setting time, compressive strength, solubility, cytotoxicity, implantation in bone, and a usage test in animals⁷⁻¹⁶. Materials, such as amalgam, Intermediate Restorative material (IRM), or SuperEBA (O-ethoxybenzoic acid), were used for comparison. The sealing ability of MTA was superior to that of amalgam and SuperEBA in both dye, bacterial, and endotoxin leakage methods, and was not adversely affected by blood contamination^{5,7,9,16}. The marginal adaptation of MTA was better than that of amalgam, IRM, and SuperEBA¹⁷. The setting time of MTA was found to be less than three hours, which is much longer than that of amalgam and IRM¹¹. Compressive strength and solubility of MTA were similar to that of IRM and SuperEBA, respectively¹¹. It also has some antibacterial effects on some of the bacterial species in the oral cavity¹⁸.

The cytotoxicity of MTA was investigated by two methods, agar overlay and radiochromium release. Mineral Trioxide Aggregate was ranked less cytotoxic than IRM and SuperEBA, but more cytotoxic than amalgam in the agar overlay method. It was found to be less cytotoxic than amalgam, IRM, and SuperEBA when the radiochromium release method was used¹⁴. With implantation of materials in guinea pig mandibles and tibias, MTA was more biocompatible than other test materials¹². Root-end fillings or furcation perforations repaired with MTA or amalgam placed in the teeth of dogs and root-end filling in monkeys were examined histologically^{8,10,19}. There was less inflammation around the root ends filled with MTA, and there was evidence of healing of the surrounding tissues. In addition, with the longer-term teeth filled with MTA, new cementum was found on the surface of the material when used as a root-end filling or furcation perforation material; this was not the case with amalgam. Based on these studies, it appears that MTA is an alternative material to be currently used for root-end fillings.

Since its introduction, numerous studies have been published regarding various aspects of MTA. Parirokh and Torabinejad²⁰ conducted a search of the literature regarding the chemical and physical properties and antibacterial activity of MTA from November 1993 to September 2009. Their search showed that there are many published reports regarding the properties of MTA. It has a long setting time, high pH, and low compressive strength. It possesses some antibacterial and antifungal properties depending on its powder-to-liquid ratio. Based on their search, they concluded that MTA is a bioactive material that influences its surrounding environment. In the second part of their review²¹ searched for the sealing ability and biocompatibility of MTA and reported that MTA seals well and is a biocompatible material. In the third part of their literature review Parirokh and Torabinejad²² examined the clinical applications of MTA in experimental animals and humans, as well as its drawbacks and mechanism of action of MTA from November 1993 to September 2009. Their search of the literature shows that MTA is a promising material for root-end filling, perforation repair, vital pulp therapy, and apical barrier formation for teeth with necrotic pulps and open apices. Another recent clinical application of MTA is its use for regenerative endodontic procedure (REP)²³. MTA has had great success in REP. According to a systematic review, approximately 85% of all REP-treated teeth were restored with MTA²³.

Regarding its mode of action, it appears that hydroxyapatite crystals form over MTA when it comes in contact with tissue synthetic fluid. This can act as a nidus for the formation of calcified structures following the use of this material in endodontic treatments. Based on the available information at that time, they concluded that MTA is the material of choice for sealing the pathways of communication between the root canal system and its external surfaces.

In two separate updated overview of the literature Parirokh and Torabinejad^{24,25} conducted another search of the literature from October 2009 to October 2016 regarding the properties and clinical uses of MTA and other bioactive endodontic cements (BECs). Based on their first search they concluded that MTA has been extensively used as a vital pulp treatment material and in most studies, newly introduced materials have been compared to MTA. Some of the BECs have shown promising results; however, the number of their studies compared to investigations on MTA is very limited. Based on the results of the second review published in 2018 they concluded that MTA has been used extensively for various clinical applications. The results of these studies showed a successful outcome when MTA was used for perforation repair, root-end filling, repair of resorptive defect and placement of apical plug in teeth with necrotic pulps and open apices. The number of publications using BECs for various clinical applications such as placement as an apical plug, perforation repair, root-end filling, coronal plug for regeneration and treating resorption defect was very limited. Furthermore, most studies had several methodological shortcomings and low levels of evidence.

According to numerous studies^{10,12,14,19;26-28,29}, MTA is superior or equivalent to other materials in terms of biocompatibility, sealing ability, and bactericidal properties. Particularly from a biological perspective, MTA has a great advantage over other available materials. In addition to its excellent biocompatibility, MTA comes close to being an ideal filling material, as it has the capacity to promote bone, dentin, and cementum regeneration.

Most dental filling materials are designed to exhibit the best results when they set in a dry environment or when moisture is properly controlled. MTA consists of hydrophilic powder and sets under wet conditions, except in environments with excessive bleeding or water. This property is advantageous in most surgical conditions because it is almost impossible to obtain a completely dry environment, despite efforts to control hemorrhage with pressure and cotton pellets.

Despite these advantages, MTA has some disadvantages. They include long setting time, difficult handling characteristics, discoloration potential, presence of toxic elements in the material, and material's cost^{11,30,35-42,53}. The setting time of MTA is approximately 3-4 hours¹¹, which is considered to be a disadvantage in some clinical situations like surgical endodontics. The blood flow in the surgical site can cause a certain amount of cement loss³⁰. Irrigation of the operative field before the MTA has set can evacuate some of the material thereby requiring an additional application of MTA. Therefore, there is a need for a substance to have a shorter setting time than the current MTA. To circumvent these problems, additives such as methylcellulose, calcium chloride, and dibasic sodium phosphate were employed to shorten its original setting time³¹⁻³³ However, the mixing of MTA powder with other substances may possibly alter the properties of MTA. Adding calcium chloride solution might lower the final compressive strength relative to that of a mixture with sterile water³⁴. Some investigators believe that handling of MTA is not simple for some of the clinical applications and require practice^{35,36}. Therefore, there is a need for a substance and a method to improve handling properties of the current MTA.

Another disadvantage of MTA is its discoloration potential. The use of MTA has been long associated with crown discoloration and poor esthetics³⁷⁻⁴². This drawback is mainly attributed to two factors: the presence of bismuth oxide⁴³, and the disintegration of the powder and release of ferrous oxide (FeO)⁴⁴. Exposure of bismuth oxide to sodium hypochlorite or blood can also lead to discoloration^{45,46}. In response to the poor esthetics associated with grey MTA, white MTA was developed with reduced amounts of carborundum (Al₂O₃), FeO, and periclase (MgO)⁴⁷. However, white MTA (WMTA) produces similar levels of discoloration^{39,42,45,38,48}. The mechanism of discoloration associated with WMTA is similar to that of grey MTA. Chrepa et al. have stated that the use of MTA in REPs is the most significant predictor of crown discoloration and increased the odds by 21 times compared to another calcium silicate called Biodentine³⁷. The use of zirconium oxide in Biodentine and Endosequence Root Repair Material (ES RRM putty) has resolved the esthetic challenge associated with original MTA⁴⁹. However, when in contact with blood, all materials exhibit variable degrees of discoloration⁴⁶. Therefore, there is a need to change the radio pacifier of the current MTA.

Portland cement is one of the main components of MTA. MTA has been suspected to be composed of undesirable elements such as arsenic. In an investigation on arsenic release from MTA, Duarte at el. showed that the amount of arsenic release is very low; thus, there is no contraindication for the use of MTA in terms of the presence of arsenic⁵⁰. However, following questioning the method and the results of that study⁵¹ recently another research study assessed the whole amount of arsenic in GMTA, WPC, grey PC (GPC), WMTA-Angelus, GMTA-Angelus, MTA-Obtura (WAMTA with a proprietary viscous liquid) and CPM (the Argentinean form of MTA)⁵². The investigators reported that the amount of arsenic is higher than the standard amount of the material (2 mg/kg) in all of the tested materials except for WPC, WMTA-Angelus, and MTA Obtura. The amount of arsenic in GPC is reported six times more than GMTA. Bramante et al.⁵² believe that because of the use of small amounts of the material for clinical applications, there is no cause for concern, even with regard to GPC. Moreover, ferric oxides can stabilize arsenic content and are frequently used for removing arsenic compounds from water⁵³. Therefore, because of the presence of ferric oxide in GMTA^{47,54} and the insolubility of the material^{11,55,56,57}, arsenic cannot be released to the tissue fluid, and therefore there is not a reason for concern.

### Methods for Placement of MTA⁵⁸

Mineral Trioxide Aggregate is difficult to deliver to a small cavity for root-end filling because its physical properties differ from those of other root-end filling materials. For delivery of the MTA, most clinicians use a syringe-type carrier or MTA pellet forming block.

Carrier- and syringe-type devices that are most often used for the delivery of MTA include: the Retro Amalgam filling carrier (Moyco Union Broach, York, PA, USA), the Messing Root Canal Gun (R. Chige, Inc., Boca Raton, FL, USA), Dovgan MTA Carriers (Quality Aspirators, Duncanville, TX, USA), the MTA Carrier (G. Hartzell & Sons, Concord, CA, USA), MAP System (PD, Vevey, Switzerland), and the Central Syringe (Centrix, Inc., Shelton, CT, USA). These carrier- and syringe-type devices have several limitations. When the root-end cavity preparation is small, the carrier-type devices can be difficult to use. If a large amount of MTA is delivered to the root-end cavity, the excess MTA is deposited onto bone cavity. If these devices are not cleaned immediately after use, the syringe can become clogged and have to be thrown out.

Lee⁵⁹ first introduced an MTA block, which was made by cutting a groove into a 0.5 inch×0.5 inch×2 inch plastic block (Tap Plastics, San Rafeal, CA, USA) using a #169 fissure bur. Using this block, the MTA is mixed to a putty-like consistency and is immediately placed into the grooves of MTA block using a cement spatula, and the excess material outside of the grooves is wiped clean. Then, the MTA pellet is scooped out of the grooves using the Lee carver (G. Hartzell & Sons, Concord, CA, USA) and delivered to the root-end preparation. The MTA pellets should be placed into the root end cavity preparation as quickly as possible because the small pellets will quickly dehydrate. When the MTA mixture is dry, it becomes crumbly and unmanageable. Covering the plastic block with moistened gauze will help to prevent desiccation of the MTA^{6,59}.

### Preferred Characteristics of Root End Filling Materials

A preferred root-end filling material should be easy to manipulate, radio pacifier, dimensionally stable, bactericidal or bacteriostatic, non-resorbable, and unaffected by the presence of moisture. It should also adhere to the prepared cavity walls, seal the root canal system, promote healing, and it should be non-toxic and well-tolerated by periapical tissues. Many studies have examined the sealing ability and bio-compatibility of root end filling materials, but none of these root-end filling materials, including MTA, fully satisfy these requirements⁶⁰. Based on available information regarding root end filling materials and techniques, there is a need for a substance to replace MTA and many other calcium silicate cements in the market that do not have these shortcomings and have shorter and variable setting time, have no discoloration potential, and a device for easier handing of these materials.

### Mechanical Aspects

Powder and liquid often need to be mixed to form a dental material (paste) for filling root canals and for other purposes. For example, water and powder may be in 3:7 ratio. As referred to herein, “powder” may generally refer to cementitious granular material that may form a paste when mixed with water. For example, a flowable suspension, or slurry in some cases, formed in a liquid matrix may be referred to as paste. It is understood that granules may be solid, soft matter, and/or other types of matter amenable of forming a paste by use of liquids. It is understood that embodiments disclosed herein may be used, or be adapted to be used, for mixing different liquids, or soft-matter, together, i.e. without necessarily including a powder. Liquids may include water and/or other solvents, suitable for achieving desired paste composition and properties. The liquid may be purified water, for example.

An exemplary mixing procedure to mix powder and liquid involves using a stick or paddle on a glass plate, e.g. analogous to mixing timed epoxy (such as 5-minute epoxy) on a piece of paper with a skewer or toothpick. Mechanical mixing and scraping may be required to ensure proper mixing, e.g. without active stirring the initial materials may remain unmixed.

For mixing of dental powders with water (the liquid), it is found that if the mixing process is not thorough enough, the resulting paste would be non-homogenous or non-uniform, e.g. areas of stagnation with no mixing may be formed. It is further found that dental paste, even if well-mixed, may be prone to separating once formed, i.e. separating into a liquid (or liquid-dominant) phase and a powder/solid (or powder/solid dominant) phase. For example, it is found that upon application of high shear or high pressure, e.g. through extrusion through an aperture of a nozzle or small orifice, the water in the dental paste may be drawn out of the aperture and wet powder may be left behind within the body of the nozzle. It is further found that, to achieve appropriate consistency of the paste to achieve target objectives, the liquids and solids may need to mixed within relatively narrow ranges. It is further found that the paste may dry quickly, thereby making the process of mixing and using the paste time-sensitive. For example, a user may need to use the paste within a predetermined amount of time within formation thereof to ensure application of wet paste. Additional constraints are imposed on the user preparing and using the paste because, due to application of paste within the oral cavity, it is important for the powder and liquid to be sterile. For example, a user may require specialized tools that are sterilized and “single-use”. In many cases, manual preparation of dental pastes may be onerous and risky. In some cases, use of amalgamators may reduce human workload and improve consistency. However, amalgamators only assist with mixing and do not provide broader advantages important for reducing user fatigue and improving outcomes. Greater improvement is desired.

### SUMMARY OF THE INVENTION

In one aspect, the present invention provides an improved MTA powder composition that produces a filling material paste when mixed with water for use in general dentistry or endodontics to replace natural tooth material.

In some embodiments, the present invention provides an improved MTA powder composition that produces a filling material paste when mixed with water for use in general dentistry or endodontics to replace natural tooth material, the composition comprising: Portland cement; extrinsic calcium sulfate as a setting enhancer; and a radio pacifier material.

In some embodiments, the improved MTA powder composition may comprise 60-70% by weight of the Portland cement; 10-15% by weight of the extrinsic calcium sulfate; and 15-20% by weight of the radio pacifier material.

In some embodiments of the improved MTA powder composition, the radio pacifier material is selected from one or more of barium titanate, bismuth oxide, and zirconium oxide. In some embodiments the radio pacifier material is barium titanate.

In some embodiments of the improved MTA powder composition, the Portland cement component of the composition may comprise: 45-80% by weight of the Portland cement of Tri Calcium Silicate; 10-25% by weight of the Portland cement of intrinsic Calcium Sulfate; 5-15% by weight of the Portland cement of Hibschite (Al₂Ca₂(SiO₄)2.16(OH)3.36); and 5-15% by weight of the Portland cement of Calcium Aluminum Oxide Carbonate (Ca₄Al₂O₆CO₃·11H₂O).

In some embodiments, the improved MTA powder composition may comprise 55-75% by weight of the Portland cement; 15-25% by weight of the extrinsic calcium sulfate; and 10-20% by weight of the radio pacifier material consisting of barium titanate. Such embodiments may be suitable for use as a filling material for pathogenic and iatrogenic tooth cavities.

In some embodiments, the improved MTA powder composition may comprise 65-85% by weight of the Portland cement; 0-10% by weight of the extrinsic calcium sulfate; and 15-25% by weight of the radio pacifier material consisting of barium titanate. Such embodiments may be suitable for use as a sealer material for natural occurring tooth cavities or root canals.

In some embodiments, the improved MTA powder composition may comprise 65-85% by weight of the Portland cement; 1-10% by weight of the extrinsic calcium sulfate; and 15-25% by weight of the radio pacifier material consisting of barium titanate.

In another aspect, the present invention provides a kit to produce a cementitious paste filler for use in general dentistry or endodontics to replace natural tooth material, the kit may comprise: a first capsule containing an amount of a powder composition of the present invention; a second capsule containing a volume of water; and instructions to mix the volume of water with the amount of the powder composition to form the cementitious paste filler.

In some embodiments of the kit, the ratio of the weight of the powder composition in the first capsule to the weight of the water in the second capsule is in the range of 3:1-3.

In another aspect, the present invention provides a method of filling a tooth cavity, comprising the steps of: identifying the cavity of the tooth to be filled; mixing a weight of the powder composition of the present invention with a volume of water to produce a filling material paste; introducing the filling material paste into the tooth cavity; and allowing the filling material paste to harden. Preferably, the time for the filler material to harden is less than 10 minutes.

In some embodiments of the method, the ratio of the of the weight of the powder composition to the weight of the water is in the range of 3:1-3.

The present invention provides cementitious filling materials to form an improved seal against migration of bacteria and provides a superior therapy for cavities found in the teeth of mammals, including humans, as well as other vertebrates.

The suitability of a particular cement composition for a given purpose is typically determined by a combination of its chemical composition and its physical attributes, i.e. the manner and degree to which the cement is ground (granulation) and the resulting particle size. Cement compositions that have a higher fineness (i.e. higher ratio of the cement's particle surface area to its weight) are faster setting cements.

The cement composition comprises a powder consisting of fine particles which are hydrophilic and which set in the presence of moisture. Hydration of the powder results in a colloidal gel which solidifies to a hard rock-like substructure in less than 10 minutes. The setting times may be shortened by increasing the calcium sulfate in the overall composition by increasing the amount of extrinsic calcium sulfate in the composition, or vice versa.

The characteristics of the cement composition depend upon the size of the particles, the powder-water ratio, temperature, presence of water, and entrained air. After setting, the composition has compressive strength equal to that of amalgam.

The improved MTA powder composition is combined with water to form the filler material paste of the present invention. Depending on the particular application, various amounts of water may be utilized to form the filler material paste. Enough water is added to produce a filler material with a putty consistency, which then solidifies to a rock-like hardness.

The fact that water is the principal reactant in the hardening reaction offers a significant advantage over many of the filling materials commonly used by allowing the cement to set in the moist environment of the body. Portland cement hardens by reactions with water, which are called hydration reactions. These reactions are complex, but principally involve the reaction of tricalcium silicate (3CaO·SiO.sub.2) and dicalcium silicate (2CaO·SiO.sub.2) with water. When these compounds react with water during the hardening of the cement, the principal hydration product is tricalcium silicate hydrate. The tricalcium silicate hardens rapidly and is most responsible for the early strength of Portland cement. The dicalcium silicate has a slower hydration reaction and is mainly responsible for strength increases beyond one week. Tricalcium aluminate, which plays a lesser role in the hardening process, hydrates rapidly also and contributes to early strength of development.

Portland cement is not radiopaque, but a radio pacifier component may be added to render it radiopaque for purposes of dental diagnostics. Bismuth oxide (Bi₂O₃) has been found to be a suitable such compound. However, the material causes tooth discoloration. A preferred radio pacifier material is barium titanate because it is biocompatible and does not cause tooth discoloration. Depending on the degree of radiopacity desired, various ratios of additive may be used. In the preferred form of the composition, the amount of the radio pacifier material may be in the range of 10-25% by weight of the total dry composition.

In a mechanical aspect, the disclosure describes a device for forming a paste from a powder and a liquid. The device includes a chamber for mixing the powder and the liquid. The chamber may be capable of isolating the powder and the liquid. In use, the device may be actuatable to intermingle the powder and the liquid to generate the paste. The device may include mixing means to mix the powder and the liquid to form the paste. The device may include an opening for drawing out the paste once prepared. The device may be electrically powered or hand powered, and/or at least partially automatically actuated or manually actuated.

It is understood that, in some embodiments, aspects disclosed herein may be used to mix liquids, soft matter, and/or solids, in a variety of combinations. For example, in some embodiments, a first liquid (or soft matter) and a second liquid (or soft matter) may be mixed together to form an emulsion. Such an emulsion may be a paste or behave as a liquid.

In an aspect, the disclosure describes a handheld device for forming a cementitious paste from a powder and a liquid. The handheld device also includes a chamber suitable for receiving the powder and the liquid and for mixing the powder and the liquid to form the cementitious paste, the chamber defining an extrusion end for drawing out the cementitious paste. A puncturing end of the device, component of the device, or other object may be drivable into the chamber to puncture a diaphragm or other barrier separating the powder and the liquid in the chamber. A mixing member may extend into the chamber and be rotatably drivable for mixing the powder and the liquid in the chamber.

In some embodiments, a nozzle is coupled to the extrusion end of the chamber to receive the paste to form a stream of the paste. In some embodiments, the nozzle may comprise one or more sequential sections. A first section of the sequential sections may have a larger diameter than a second section of the sequential sections. In some embodiments, the nozzle may comprise one or more tapered sections defined by a tapering diameter towards an exit end of the nozzle. In some embodiments, tapered sections may be defined by a constant angle of taper.

In some embodiments, the nozzle is coupled to a vibrator to vibrate the stream. In some embodiments, the vibrator is at least one of a sonic or ultrasonic vibrator.

In some embodiments, the puncturing end is an end of a needle. The needle may be coupled to a barrel containing the liquid. A plunger may be disposed within the barrel. In some embodiments, the plunger disposed within the barrel is actuatable to inject the liquid into the chamber to permit mixing of the liquid and the powder to form the paste.

In some embodiments, the chamber is tubular, and the puncturing end is an end of a puncturing member disposed at least partially coaxially with the chamber. In some embodiments, the mixing member includes a coupler arranged at least partially coaxially with the chamber. The puncturing member may be disposed inside the coupler and may be movable within the coupler to be releasable into the chamber via an end of the coupler disposed in the chamber. In some embodiments, the diaphragm seals an opening at the end of the coupler. The puncturing member may be movable within the coupler to puncture the diaphragm to be released into the chamber. In some embodiments, the coupler is rotatable relative to the puncturing member at least partially coaxially with the chamber. In some embodiments, an outer end of the coupler is radially tapered. The coupler may define a gasket abutting an end of the chamber to sealably close the end of the chamber.

In some embodiments, the diaphragm is a membrane of a capsule. In some embodiments, the membrane is an outer covering of the capsule. In various embodiments, the capsule may contain liquid and/or powder. In some embodiments, the capsule may define two isolated volumes, one each for the liquid and the powder. In some embodiments, the capsule is one of two capsules that includes a first capsule containing liquid and a second capsule containing powder. The capsule may be disposed inside the chamber.

In some embodiments, the diaphragm is coupled to the chamber to separate the chamber into two separate portions or volumes.

In some embodiments, the mixing member comprises a rotatable central shaft disposed in the chamber and a plurality of members extending radially from the central shaft to provide agitation to mix the powder and the liquid when the central shaft is being rotated. The rotatable central shaft may be rotatable in one and/or two rotational directions, e.g. to create back and forth agitation. The device may be operable to rotate the central shaft in a selectable direction. The device may be operable to rotate the central shaft in sequentially alternating directions. In some embodiments, a first member of the mixing member extends parallel to the central shaft and is radially spaced apart from the central shaft. A second member of the mixing member may be opposed the first member extending parallel to the central shaft. The second member may be spaced apart from the central shaft. The first member and the second member may be rotatable to cause whisking of the powder and the liquid.

In some embodiments, the mixing member includes a spring engaged with the chamber. In some embodiments, the chamber is tubular. The mixing member may include a coil spring at least partially coaxially arranged within the chamber. In some embodiments, the coil spring abuts an inner wall of the chamber. In some embodiments, the coil spring is compressed to frictionally engage with an end of the chamber. In some embodiments, the mixing member includes a coupler frictionally engaged with the coil spring to allow rotation of the coil spring by rotation of the coupler. The coupler may extend at least partially coaxially at least partially within the chamber. The coupler may be suitable for coupling to a driver.

In some embodiments, the chamber is tubular. The mixing member may include a mixing end that extends at least partially coaxially along, and within, the chamber for rotation within the chamber. In some embodiments, the mixing member includes a coupler connected to the mixing end to allow rotation of the mixing end by rotation of the coupler. The coupler may extend at least partially coaxially at least partially within the chamber. The coupler may be suitable for coupling to a driver. In some embodiments, the driver is an electric motor.

In some embodiments, the coupler defines a front face disposed within the chamber and a rear face accessible from outside the chamber. In some embodiments, the front face forms an end of the chamber. In some embodiments, the mixing end is a coil spring, and the front face is frictionally engaged with the mixing end. In some embodiments, the rear face defines a coupling surface for coupling with a driver. In some embodiments, the coupler is suitable for coupling with the driver by circumferential engagement of a radially outer surface of the coupler with a radially inner surface of the driver.

In some embodiments, the chamber includes an opening for receiving the liquid. In some embodiments, the opening is coaxial with the mixing member. The opening may be coaxial with a puncturing member defining the puncturing end. In some embodiments, the opening opens at least partially lateral to a central axis of the chamber.

In some embodiments, the chamber includes an agitating ball for mixing the powder and the liquid when the chamber is shaken.

In some embodiments, the diaphragm may be a first diaphragm. A second diaphragm may seal the chamber. The second seal may be spaced apart from the first seal. The puncturing end may be capable of puncturing the second diaphragm. The first and second diaphragm and the puncturing end may be arranged around a common axis such that the second diaphragm may be punctured only after puncturing the first diaphragm. After mixing, a user may push the puncturing end in to puncture the second diaphragm to release the paste out of the extrusion end. In an aspect, the disclosure describes a method of generating a stream of cementitious paste using powder and liquid. The method may include puncturing a diaphragm in a chamber to intermingle the powder and the liquid. The method may include rotatably driving a mixing member in the chamber to mix the powder and the liquid to form a cementitious paste. The method may include extruding the cementitious paste through an opening in the chamber to form the stream of the cementitious paste.

In some embodiments, the method includes receiving the powder and the liquid into the chamber. In some embodiments, the method includes isolating the powder and the liquid. In some embodiments, isolating the powder and the liquid involves using a diaphragm to separate the powder and the liquid.

In an aspect, the disclosure describes a handheld device for forming a cementitious paste from a powder and a liquid. The handheld device includes a chamber defining an extrusion end for drawing out the cementitious paste. The handheld device may include a diaphragm disposed in the chamber to separate the chamber into a first volume for containing the powder and a second volume for containing the liquid. The diaphragm may be suitable for isolating the powder and the liquid. A magnetic mass may be disposed inside the chamber to provide agitation. In various embodiments, the magnetic mass may be a permanent magnetic or a ferromagnetic metal such as iron, nickel, cobalt, steel, or other materials (e.g. alloys) that may be attracted or repulsed by a magnet (are capable of being magnetically actuated). The magnetic mass may be actuatable by one or more electromagnets. When actuated, the magnetic mass may be operable to move within the chamber to puncture the diaphragm and to mix the powder and the liquid.

In some embodiments, the magnetic mass may have a surface suitable for abrasively puncturing the diaphragm and mixing the powder and the liquid. The magnetic mass may include surface protrusions. The magnetic mass may have a textured surface. The magnetic mass may have a roughened surface.

In some embodiments, the one or more electromagnets includes a plurality of coils at least partially surrounding (externally) the chamber to generate a magnetic field inside the chamber. In some embodiments, a first current passes through a first coil and a second current passes through a second coil. The first and second current may be adapted to generate a time-varying magnetic field that causes substantially continuous agitation of the powder and the liquid inside the chamber by magnetically actuating the magnetic mass by the time-varying magnetic field. In some embodiments, the one or more electromagnets may be configured to generate a magnetic field inside the chamber to cause reciprocating motion of the magnetic mass.

In some embodiments, the magnetic mass may be disposed inside the first volume before puncturing the chamber.

In various embodiments, the one or more electromagnets include a first electromagnet and a second electromagnet, the first electromagnet and the second electromagnet being at least partially circular and spaced apart from each other, the chamber being tubular and extending from within the first electromagnet to within the second electromagnet. The magnetic mass may be magnetically actuated by the one or more electromagnets to move between the first electromagnet and the second electromagnet within the chamber.

In an aspect, the disclosure describes a method of generating a stream of cementitious paste using powder and liquid. The method includes moving a magnetic mass in a chamber by magnetic actuation. Moving the magnetic mass may puncture a diaphragm in the chamber to intermingle the powder and the liquid. After intermingling of the powder and the liquid, moving the magnetic mass in the chamber by magnetic actuation may mix the powder and the liquid to form a cementitious paste. Extruding the cementitious paste through an opening in the chamber may form the stream of cementitious paste.

In an aspect, the disclosure describes a handheld device for mixing powder and liquid to form a cementitious paste. The handheld device includes a holder for receiving a capsule containing the powder and the liquid. The handheld device may include a base. The handheld device may include one or more linear actuators coupling the base to the holder. The one or more linear actuators may be actuatable to shake the holder to mix the powder and the liquid inside the capsule. In some embodiments, a non-magnetic mass may be included in the capsule to improve mixing during shaking. In some embodiments, the capsule may contain a spiked, mace-shape, or otherwise abrasive mass (or any mass particularly conducive to cause concentration of force to facilitate puncturing) to puncture the diaphragm and/or membrane inside the capsule.

In an aspect, the disclosure describes a method of mixing powder and liquid. The method includes receiving a capsule containing the powder and the liquid in a holder. The method may include actuating a plurality of linear actuators coupled to the holder to shake the holder so as to mix the powder and the liquid inside the capsule.

It is understood that, in various embodiments, capsules, the mixing chamber, injectors, handpieces, and/or other components may be provided separately, in combination, and/or in a plurality of combinations.

Embodiments can include combinations of the above features.

Further details of these and other aspects of the subject matter of this application will be apparent from the detailed description included below and the drawings.

### BRIEF DESCRIPTION OF THE DRAWINGS

FIG. 1 is a scanning electron microscope (SEM) image of particles from a sample of a preferred embodiment of the composition of the present invention;

FIG. 2 shows photographs of five representative samples of the results in a leakage study conducted on a preferred embodiment;

FIG. 3 shows radiographs of teeth in live dogs that have had cavities filled with a filling material made from an embodiment of the present invention;

FIG. 4A is a schematic cross-sectional view of a handheld device powered by an electric motor, in accordance with an embodiment;

FIG. 4B is a schematic cross-sectional view of a handheld device powered by hand, in accordance with another embodiment;

FIG. 5A is a side elevation view of a handheld device, in accordance with an embodiment;

FIG. 5B is a cross-sectional view of the handheld device of FIG. 5A, in accordance with the embodiment;

FIG. 5C is a perspective view of the handheld device of FIG. 5A, in accordance with the embodiment;

FIG. 6A is a cross-sectional view of a handheld device, in accordance with an embodiment;

FIG. 6B is a perspective view of the handheld device of FIG. 6A, in accordance with the embodiment;

FIG. 7 is a schematic cross-sectional view of a handheld device, in accordance with the same or another embodiment;

FIG. 8A is schematic cross-sectional view of a mixing member disposed in a chamber, in accordance with an embodiment;

FIG. 8B is schematic cross-sectional view of a mixing member disposed in a chamber, in accordance with another embodiment;

FIG. 8C is schematic cross-sectional view of a mixing member disposed in a chamber, in accordance with another embodiment;

FIG. 9 is schematic cross-sectional view of a mixing member disposed in a chamber having an opening for liquid, in accordance with another embodiment;

FIG. 10 is schematic cross-sectional view of a mass disposed in a chamber for mixing, in accordance with another embodiment;

FIG. 11A is a partial sectional view of a handheld device disposed inside a magnetic actuator, in accordance with another embodiment;

FIG. 11B is a perspective partial sectional view of the handheld device of FIG. 11A, in accordance with the embodiment;

FIG. 12 is a perspective view of a device for mixing liquid and powder, in accordance with an embodiment;

FIG. 13A is a schematic side-view of a device for generating a paste using liquid and powder during a first step, in accordance with an embodiment;

FIG. 13B is a schematic side view of a device for generate the paste during a second step, in accordance with an embodiment;

FIG. 14 is a perspective view of a nozzle couple to a device and a vibrator, in accordance with one or more embodiments;

FIG. 15 illustrates a block diagram of a computing device, in accordance with one or more embodiments;

FIG. 16A is a schematic sectional view of a chamber with a diaphragm separating the powder and the liquid, in accordance with an embodiment;

FIG. 16B is a schematic sectional view of a chamber with two capsules disposed therein, in accordance with another embodiment;

FIG. 16C is a schematic sectional view of a chamber with a capsule disposed therein, in accordance with another embodiment;

FIG. 17A is a cross-sectional view of a nozzle and a forward portion of a plunger, in accordance with an embodiment;

FIG. 17B is a cross-sectional view of the nozzle and the forward portion of the plunger of FIG. 17A with the plunger engaged with the nozzle, in accordance with an embodiment;

FIG. 18A is a cross-sectional view of a nozzle and a forward portion of a plunger, in accordance with another embodiment;

FIG. 18B is a cross-sectional view of a nozzle and a forward portion of a plunger, in accordance with yet another embodiment; and

FIG. 18C is a cross-sectional view of a nozzle and a forward portion of a plunger, in accordance with a yet further embodiment.

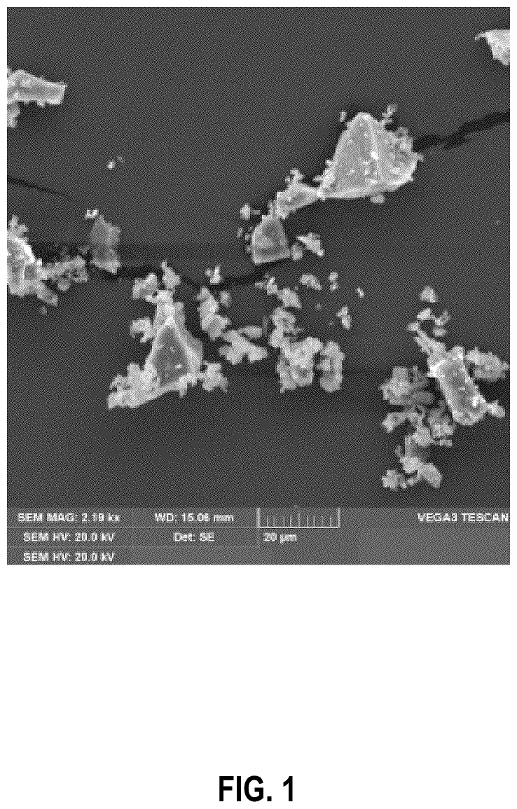

### DETAILED DESCRIPTION

As noted above, the present invention provides improved MTA cementitious filling materials for use in dentistry that set more rapidly than the prior art MTA materials to form an improved seal against migration of bacteria. This provides an improved therapy for cavities found in the teeth of mammals, including humans, as well as other vertebrates.

Typically, Portland cement is combined with aggregate and water to form concrete. The cement and water coat and bind to the aggregate, filling the spaces between the aggregate particles to form a ceramic composite material. The cement composition of the present invention, however, does not require the use of aggregate, but utilizes Portland cement in combination with extrinsic calcium sulfate as a hardening/setting enhancer, with water. Portland cement contains an intrinsic amount of calcium sulfate; however, the addition of extrinsic calcium sulfate greatly reduced the setting time of the composition while producing a filling material that exhibits many of the desired characteristics of dental filling materials.

The process of making Portland cement is well known and it can be purchased from any number of manufacturers under various trade names. The basic raw materials for Portland cement are lime (CaO), silica (SiO.sub.2), alumina (Al.sub.2 O.sub.3) and iron oxide (Fe.sub.2 O.sub.3). These components are appropriately proportioned to produce various types of Portland cement.

The preferred embodiment of the present invention utilizes a Type I Portland cement having the following approximate composition:

| Component | percentage |
|---|---|
| CaO | 70.4 |
| SiO2 | 13.6 |
| SO3 | 10.9 |
| Al203 | 3.7 |
| MgO | 0.5 |
| Fe203 | 0.3 |
| K2O | 0.2 |
| Na20 | 0.2 |
| SrO | 0.1 |

This preferred Portland cement is commercially available as the White Portland Cement Manufactured by Chromacrete and distributed by PCS Industries in San Diego California.

The suitability of a particular cement composition for a given purpose is typically determined by a combination of its chemical composition and its physical attributes, i.e. the manner and degree to which the cement is ground (granulation) and the resulting particle size. In this formulation, the cement composition comprises a powder consisting of fine particles which are hydrophilic and which set in the presence of moisture. Hydration of the powder results in a colloidal gel which solidifies to a hard rock-like substructure in less than four hours. The characteristics of the cement composition depend upon the size of the particles, the powder-water ratio, temperature, presence of water, and entrained air. After setting, the composition has compressive strength equal to that of amalgam.

Preferably, the particles in the powder composition have a maximum diameter of less than 50 μm. More preferably, 95% of the particles in the powder composition have a maximum diameter of less than 10 μm, and even more preferably 87% of the particles in the powder composition have a maximum diameter of less than 5 μm.

### Application 1: Filling Material for Pathogenic and Iatrogenic Tooth Cavities:

An embodiment of an improved MTA powder composition in accordance with present invention was produced with a starting composition of white Portland cement powder manufactured by Chromacrete. To this was added calcium sulfate as a hardening/setting enhancer, and barium titanate as a radio pacifier material to provide radiopacity to the filling material. The proportions by weight of the composition were as follows:
55-75% Portland cement;
15-25% extrinsic calcium sulfate; and
10-20% barium titanate.

The composition was determined to comprise the following by weight of the total composition:
45-80% Tri Calcium Silicate
10-25% Intrinsic Calcium Sulfate
5-15% Hibschite—Al2Ca2(SiO4)2.16 (OH)3.36
5-15% Calcium Aluminum Oxide Carbonate—Ca4Al2O6CO3·11H2O

SEM particle size analysis and aspect ratio analysis was conducted of a sample of this embodiment for non-spherical particles, the particle size derived by image processing is represented by the equivalent diameter D. The equivalent diameter is the calculated diameter that corresponds to a hypothetical sphere that has the same surface area as the particle analyzed in the sample. The results are shown Table 1.

TABLE 1
Particle size distribution of a preferred embodiment.

| Particle Size Range (μm) | Percentage in the size |
|---|---|
| see Note 1 | range (number %) |
| D < 5 | 86.9 |
| 5 ≤ D < 10 | 8.7 |
| 10 ≤ D < 20 | 3.7 |
| 20 ≤ D < 50 | 0.6 |
| 50 ≤ D | 0 |
| Maximum Diameter (D) (μm) | 34.9 |
| Minimum Diameter (d) (μm) | 0.47 |
| Average Diameter (μm) | 2.7 |
| Median Diameter (μm) | 1.4 |

Average: The arithmetic mean. Calculated by taking the sum of a group of numbers and then dividing by the count of those numbers.
Median: The middle number of a group of numbers

The size reported in the table is represented by the equivalent diameter D. The equivalent diameter is the calculated diameter that corresponds to a hypothetical sphere that has the same surface area as the particle analyzed in the sample.

A sample of this embodiment was mixed with water at a ratio of the weight of the powder composition to the weight of the water was 3:1.

pH:

Examination of the pH of this embodiment of the filling shows that it has an alkaline pH of 12.5, similar to the original MTA.

### Setting Time:

The setting time of this filling material after mixing was approximately 5 minutes. The setting time can be modified by increasing or decreasing of the calcium sulfate in the mixture depending on the application of the proposed filling material.

### Leakage Study:

In many dental applications, the ultimate success of the treatment often depends on the adaption of the filling material to the tooth walls, and the resultant seal between the filler and the remaining tooth structure. An ideal seal will prevent the migration of bacteria and other byproducts into the cavity. The efficiency of the seal is particularly important where the pulp chamber is to be sealed.

The clinician will recognize that adaption and sealing ability of a filling material can be measured in various ways. Any test of adaption and sealing ability attempts to determine the filling material's ability to seal the cavity from bacteria and other organisms that can promote further decay. Therefore, the filling material's effectiveness can also be directly determined by clinical studies; however, these are subject to many variables and require significant time and expense. To simulate the clinical function, the filling material can be evaluated by a dye penetration test. Various dyes have been used to measure the sealing ability of materials to tooth structure, including the use of methylene blue dye as a tracer. An operating microscope was used to determine the degree of dye penetration. This method of measuring the adaption and sealing ability is well known to the clinician^{61,62}.

In dye penetration tests, the cavity is first prepared and filled with the material to be tested. After the outside of the tooth is coated to prevent dye leakage through anywhere but the cavity being tested, the tooth is immersed in a solution of the dye. The tooth is then sectioned and examined under the operating microscope, and the degree of dye leakage along cavity walls measured. Such leakage is expressed in terms of the distance (millimeters) travelled by the dye. In one such test, root canals for ted single-rooted extracted human teeth were prepared using the standard step-back technique. The canals were obturated with gutta percha and Grossman sealer using the lateral condensation technique. The roots were then wrapped in moist gauze pads and kept in 100% humidity for a week prior to root end preparations.

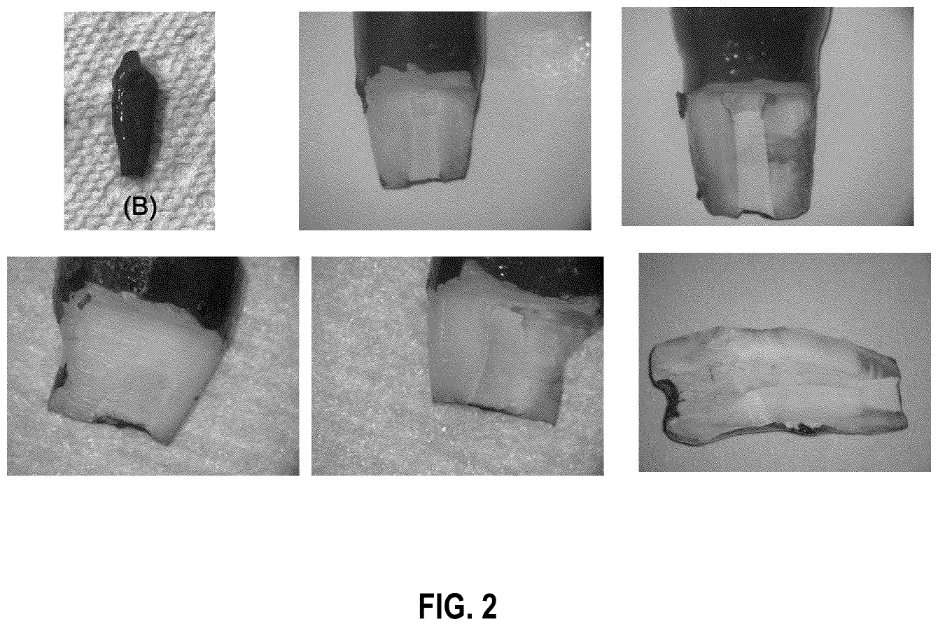

Nail varnish was then applied to the entire external surface of each root and allowed to dry. About 3-4 millimeters of the apical segment of each root was removed at a 90 degree angle to the longitudinal axis of the root. The root end cavities were filled with filling material produced in accordance with the present invention. The roots were then totally immersed in an aqueous solution of 1% methylene blue dye for 24 hours. By using a high-speed diamond burr, the apical portion of each root was ground parallel to the longitudinal axis of the tooth. The extent of leakage along cavity walls was then observed under the operating microscope. The results showed no leakage in any of the ten samples prepared, of which five representative samples are show in the photographs of FIG. 2.

### In Vitro Cytotoxicity Study: Evaluation of Cytotoxicity of Improved MTA Using the Agar Diffusion Method

A study was conducted to evaluate the cytotoxicity of Improved MTA (I-MTA) using the agar diffusion method. The test was conducted following the procedures specified in ISO 7405 (2018), ISO 10993-5 (2009) and ISO 10993-12 (2021).

Cells were mouse fibroblasts, clone of strain L (NCTC clone 929, L929, ATCC CCL-1) obtained from American Type Culture Collection (Manassas, VA). Minimum essential medium Eagle (MEM, with L-glutamine, non-essential amino acids), antibiotic antimycotic solution (with 10,000 units penicillin, 10 mg streptomycin and 25 μg amphotericin B per mL), neutral red solution, Hank's balanced salt solution (HBSS) and phenol were purchased from Sigma-Aldrich (St. Louis, MO). Fetal bovine serum (FBS) was from VWR (Radnor, PA). Agar was obtained from Becton Dickinson and Company (Franklin Lakes, NJ). Cottonseed oil was from Acros Organics (Geel, Belgium). Tissue culture flasks were from Corning Costar Corporation (Cambridge, MA). Multi-well tissue culture plates (6-well Falcon 3046) were purchased from Fisher Scientific (Waltham, Massachusetts).

The test material of the improved MTA composition (I-MTA) was stored at 22° C. until tested. The I-MTA powder tested was a combination of 65% Portland cement, 15% of calcium sulfate and 20% barium titanate. The I-MTA powder was mixed with sterile purified water at a ratio of 3:1 (w/w), then placed in a TEFLON disk mold of 6 mm in diameter and 2 mm in height for setting of 60 minutes. Both the solid material and extracts were examined for cytotoxicity using the agar diffusion method (ISO, 2018; ISO, 2009). Two types of extracts of each resin materials were prepared aseptically by incubating the samples in sterile culture medium without serum (polar solvent) or cottonseed oil (non-polar solvent) (0.2 g material per mL extractant) at 37° C. for 72 hours, following the procedures described in ISO guidelines (2018; 2009; 2021). The evaluation of extracts was to detect cytotoxicity, if any, associated with agents that may leach out from the material after the application. Sterile culture media without serum was included as the negative control and phenol served as the positive control.

L929 cells were cultured at 37° C. in a 100% humidified atmosphere with 5% CO2. At confluence, cells were washed with HBSS and harvested in culture media. The cell density was determined using an Automated Cell Counter (Countess, Invitrogen, CA) and was adjusted to 1.0×105 cells/mL. Cell suspensions were aliquoted into 6-well plates (5 mL/well) and cultured for 24 hours. The media was then withdrawn and an overlay agar (3% agar in 2× complete culture media at the ratio of 1:1), maintained at 45° C., was poured over the cell monolayer. The agar was allowed to solidify at room temperature for approximately 10 minutes, and 200 μL of neutral red solution (0.033%) was placed on the agar surface for approximately 20 minutes. The excess dye was then removed. The plates were shielded from light after the neutral red solution was added.

A volume of 50 μL extracts of the samples as well as the negative and positive controls were aliquoted onto sterile filter disks (6 mm in diameter, glass fiber prefilters, Millipore Sigma, Burlington, MA). The filter disks and the solid samples were placed at the center of the agar surfaces. Four samples were tested for each group, each in a separate 6-well plate to avoid cross contamination of materials. Plates were then returned to the incubator. Cytotoxicity was examined by measuring the zones of decolorization and evaluating cell lysis under an inverted microscope using the established criteria (Appendix A) after 24 and 48 hours.

The data obtained from the 24-hour and 48-hour examinations are presented in Tables 1 and 2, respectively. The results of the controls were consistent with the historical data of this laboratory. The positive control, phenol, caused significant cytotoxicity at both 24 and 48 hours, as evidenced by decolorization in the entire well (well diameter of 3.5 cm) and substantial cell lysis. No cell decolorization and lysis were observed in the negative control.

The I-MTA and both its MEM and cottonseed oil extracts did not induce any cell decolorization and lysis at 24-hour and 48-hour evaluation. The observations were comparable to those obtained from the negative control (Table 2).

TABLE 1
Evaluation of Cytotoxicity of Improved MTA Using
the Agar Diffusion Method (24-hour Data)

| | Decolorization | | Cell Lysis | | Cell | |
|---|---|---|---|---|---|---|
| Group ^{a} | cm ^{b} | Index | % | Index | Response | Cytotoxicity |
| Negative Control | | | | | | |
| MEM | 0 | 0 | 0 | 0 | 0 | None |
| Cottonseed Oil | 0 | 0 | 0 | 0 | 0 | None |
| Improved MTA | | | | | | |
| Set Material | 0 | 0 | 0 | 0 | 0 | None |
| Medium Extracts | 0 | 0 | 0 | 0 | 0 | None |
| CS Oil Extracts | 0 | 0 | 0 | 0 | 0 | None |
| Positive Control | 1.45 ± 0.00 | 5 | 83.0 ± 0.05 | 4.75 | 5 | Severe |

^{a} Negative Control: filter disk with 50 μL sterile culture media (MEM) without serum; CS Oil: cottonseed oil; Positive Control: filter disk with 50 μL phenol.
^{b} N = 4. The distance from the sample (cm) = (Diameter of the Decolorization Zone − Diameter of the sample)/2. The value of 1.45 cm indicates a decolorization of entire culture well (3.5 cm in dlameter): 1.45 cm × 2 + 0.6 cm (diameter of the sample). Decolorization Index is 1 if the Decolorization Zone is limited to the area under the sample (Appendix A).

TABLE 2
Evaluation of Cytotoxicity of Improved MTA Using
the Agar Diffusion Method (48-hour Data)

| | Decolorization | | Cell Lysis | | Cell | |
|---|---|---|---|---|---|---|
| Group ^{a} | cm ^{b} | Index | % | Index | Response | Cytotoxicity |
| Negative Control | | | | | | |
| MEM | 0 | 0 | 0 | 0 | 0 | None |
| Cottonseed Oil | 0 | 0 | 0 | 0 | 0 | None |
| Improved MTA | | | | | | |
| Set Material | 0 | 0 | 0 | 0 | 0 | None |
| Medium Extracts | 0 | 0 | 0 | 0 | 0 | None |
| CS Oil Extracts | 0 | 0 | 0 | 0 | 0 | None |
| Positive Control | 1.75 ± 0.00 | 5 | 100.0 ± 0.05 | 5 | 5 | Severe |

^{a} Negative Control: filter disk with 50 μL sterile culture media (MEM) without serum; CS Oil: cottonseed oil; Positive Control: filter disk with 50 μL phenol.
^{b} N = 4. The distance from the sample (cm) = (Diameter of the Decolorization Zone − Diameter of the sample)/2. The value of 1.45 cm indicates a decolorization of entire culture well (3.5 cm in diameter): 1.45 cm × 2 + 0.6 cm (diameter of the sample). Decolorization Index is 1 if the Decolorization Zone is limited to the area under the sample (Appendix A).

### APPENDIX A

### Evaluation Criteria for Agar Diffusion Test (ISO 7405, 2018)

### Decolorization Index

| Decolorization | |
|---|---|
| index | Description |
| 0 | No detectable decolorization zone around or under |
| | specimen |
| 1 | Decolorization zone limited to area under specimen |
| 2 | Decolorization zone extends <0.5 cm beyond specimen |
| 3 | Decolonization zone extends 0.5-1.0 cm beyond |
| | specimen |
| 4 | Decolorization zone extends >1.0 cm beyond |
| | specimen but does not involve entire dish |
| 5 | Decolonization zone involves entire dish |

### Lysis Index

| Lysis index | Description of decolorized zone |
|---|---|
| 0 | No observable cytotoxicity |
| 1 | <20% of the decolorized zone affected |
| 2 | 20% to <40% of the decolorized zone affected |
| 3 | 40% to <60% of the decolorized zone affected |
| 4 | 60% to <80% of the decolorized zone affected |
| 5 | >80% of the decolorized zone affected |

### Cell Response and Interpretation of Cytotoxicity

| Scale | Cell response | Interpretation |
|---|---|---|
| 0 | 0 | Non cytotoxic |
| 1 | 1 | Mildly cytotoxic |
| 2 | 2 to 3 | Moderately cytotoxic |
| 3 | 4 to 5 | Severely cytotoxic |

Based on the results of this test it appears that this combination has no toxicity and is amazingly similar to the negative control group.

### In Vivo Bio Compatibility Study:

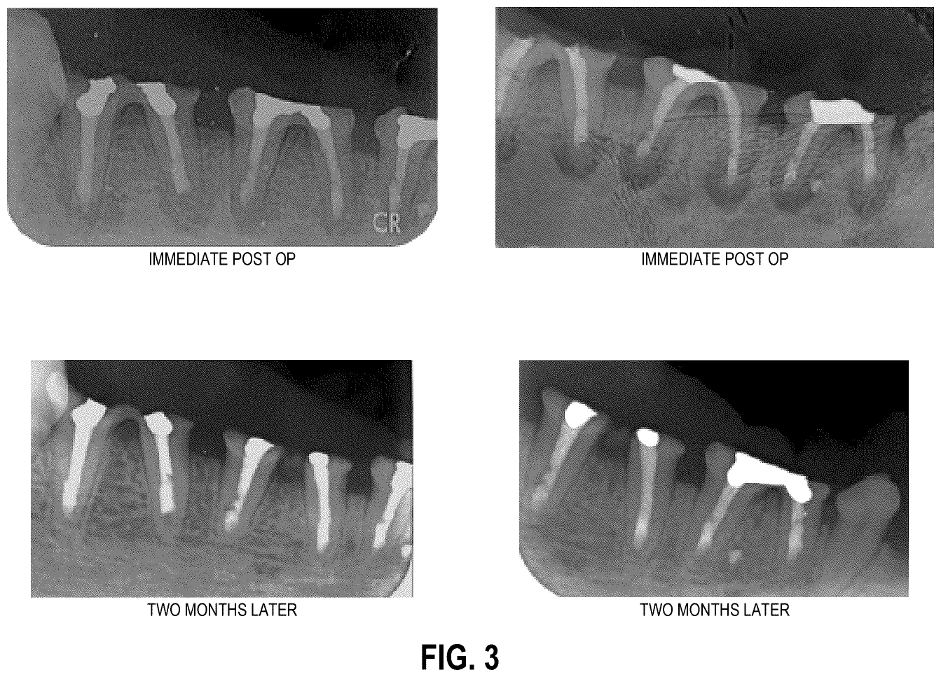

The purpose of this study was to compare the dento-alveolar and osseous healing of this embodiment of the present invention with those of the MTA as root-end filling materials in dogs. The root canals of 12 mandibular premolars in two year old dogs were obturated with gutta-percha and sealer. Two to four weeks later, during periapical surgeries, the root-end cavity preparations in these teeth were filled with either the filling material of the present invention or MTA. The material tested here was a combination of 65% Portland cement, 15% of calcium Sulfate and 20% barium titanate. Radiographic examinations after two and four months show excellent healing in both groups (FIG. 3). Based on radiographic findings it appears that this combination is very biocompatible and results in excellent healing. (FIG. 3).

### Application 2: Filling Material for Natural Occurring Tooth Spaces (Sealer):

Another embodiment of an improved MTA powder composition in accordance with present invention was produced with Portland cement powder, extrinsic calcium sulfate and barium titanate. The proportions by weight of the composition were as follows:
65-85% Portland cement;
0-10% extrinsic calcium sulfate; and
15-25% barium titanate.

A sample of this embodiment was mixed with water at a ratio of the weight of the powder composition to the weight of the water of 3:1-2. Examination of the pH of this embodiment of the filling shows that it has an alkaline pH of 12.5. The setting time of this filling material after mixing was approximately 10 minutes.

Although not all of the cement compositions falling within the ranges taught herein had been utilized in test involving the present method, it is believed that such cement compositions could be effectively utilized.

### Mechanical Aspects

There is disclosed a device operable to contain precise or predetermined volumes of concrete powder (powder) and a purified water. These materials may be provided sterile. The water and powder may be isolated from each other to prevent mixing or loss of material until actual mixing and dispensing are required, e.g. when application in a dental setting is called upon. A first chamber, space, or volume may contain the powder and a separate chamber, space, or volume may contain the water, isolated from each other.

The user may depress a button, plunger, or other mechanism to combine or intermingle the water and powder from the disparate chambers into a single chamber. In some embodiments, this may done automatically using electronics. The combining may be executed in the powder chamber, the water chamber, and/or another chamber.

After the water and powder are combined, the two materials may be thoroughly mixed. This may need to be carried out quickly before the mixture sets up. As mentioned earlier, the powder may be very fine and may not mix very easily with water. The two materials may need to be actively mixed, e.g. using a mechanical mechanism, like a paddle, propeller, whisk, ball. The materials may also be shaken violently to mix the materials.

After mixing, the material may be dispensed through a fine needle tube (18 gauge). The tub may be bent, e.g. 45 degrees, to make it easier to apply to a tooth. The tube may be constructed of metal.

In some cases, vibration may be applied to the tube tip to help pack in material and/or to help dispense material through the tube.

In some embodiments, a capsule may store the powder in the mixing chamber. For example, a new, unmixed capsule may be inserted into a handpiece that helps the user to manipulate the capsule using levers, and/or motors. Water may be stored in a separate chamber (water chamber), such as a syringe. The syringe may be depressed to inject water into the powder. Then a mixing end, e.g. a spring, a paddle, or a whisk shape, may spin at high speed, e.g. in excess of 1000 rpm in the mixing chamber. The high speed may disrupt the mixture and helps to mix thoroughly. After mixing in 10-30 seconds, a plunger may extrude the mixture out through one end of the mixing chamber. The plunger may connect to a needle tip for dispensing. For example, the connection could be a Luer™ fitting. For example, the approximate amount of material to be dispensed may be less than 1 ml.

In some cases, the capsule may be disposed of. In some cases, the tip may not be reusable because cleaning the tube may be inconvenient or difficult to properly complete.

In some cases, the spring may be permanently attached to the water chamber. Teeth on the back end of the water chamber may allow it to be rotated by a motor, e.g. an electric motor. The mixing end, e.g. spring, may also allow the user to push in the water chamber (after depressing the plunger) to dispense mixed materials.

In some cases, a foil or thin plastic seal on the end of the water chamber may be punctured by the pointed end of the plunger. The seal may ensure that no water leaks during storage and no mixing occurs until actual use. In some cases, a similar seal may exist on the powder chamber, e.g. a membrane of the capsule may provide sealing.

In various embodiments, water may be injected at the distal end, proximal end, or in the middle of the chamber.

Dispensing may be done in more than one step. For example, it may be more difficult to extrude the mixed material if the diameter difference between a first section and a second section is large. For instance, extruding by pushing from a 0.25″ diameter tube to or towards a 0.040″ diameter tube may be relatively difficult, while extruding by pushing from a 0.1″ tube to a 0.040″ tube may be relatively easy. In some cases, in order to extrude the material through a small tip, it may be necessary to dispense from the mixing chamber into a secondary chamber and then into the dispensing section. This may also require multiple plungers.

In various embodiments, the powder to water ratio may be varied to provide thinner or thicker material.

In various embodiments, a mixing mechanism with a spiral feature might be helpful to help move the material to one end to ensure mixing. Such a feature may cause mixing in one direction and dispense in the opposite.

In some cases, mixing should be sure to scrape walls of the mixing chamber and move material. It may be unacceptable to have a non-uniform mixture.

In some cases, the mixing mechanism (mixing end) could interfere with dispensing of the paste. Advantageously, a spring (e.g. coil spring) as a mixing end may compress flat to avoid interfering with dispensing of the paste. Other mechanisms such as a whisk shape may be made out of a material like nitinol so that they compress easily.

Aspects of various embodiments are described in relation to the figures.

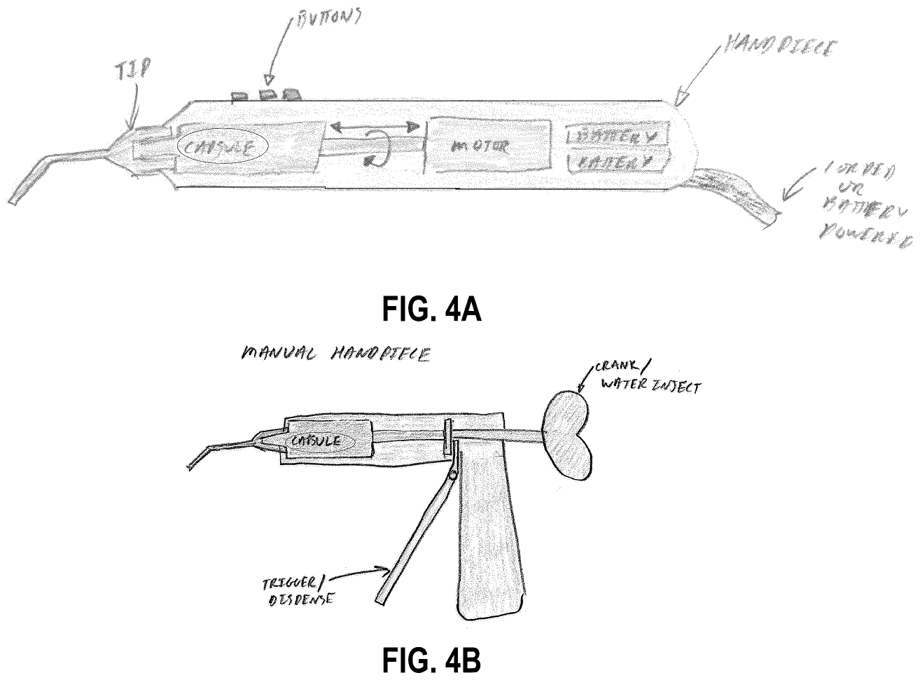

FIG. 4A is a schematic cross-sectional view of a handheld device powered by an electric motor, in accordance with the embodiment.

The handheld device may be suitable for forming a cementitious paste from a powder and a liquid.

The handheld device may be electrically powered, e.g. by a cable and/or batteries. The handheld device may comprise a handpiece configured to be held in the hand of a user. The handpiece may be adapted to or dimensioned to fit into a human hand. For example, a user may insert an end (shown bent at approximately 45° extending from the “tip”) of a nozzle into a patient's oral cavity to apply dental paste on to a tooth of the patient.

An electric motor may be disposed within the handheld device. In some embodiments, the electric motor be affixed in the handheld device to prevent relative motion between the handpiece and the electric motor.

The electric motor may be coupled to a shaft protruding into a (mixing) chamber of the handpiece for mixing the material (e.g. powder) and the liquid. The chamber may include one or more capsules that may contain the powder and the liquid. The chamber may include powder and/or liquid outside the capsule. The chamber and/or capsule(s) may be configured to isolate the powder from the liquid and to isolate the liquid from the powder. Actuation of the handheld device may disestablish isolation of materials to facilitate mixing.

In various embodiments, the shaft may rotate and/or translate for achieving mixing of powder and liquid. For example, rotation of the shaft may whisking or agitation via members extending outwardly from the shaft into the capsule. For example, translation of the shaft may ensure homogeneity of mixing and the resulting paste. In some embodiments, translation of the shaft may be limited to translation along a single axis in the handpiece, e.g. slidably translation along a long axis (or direction of elongation) of the handpiece or the capsule via prismatic joint.

In some embodiments, the sliding motion of the shaft and the rotational motion of the shaft may be achieved by separate electric motors or by the same electric motor. For example, the shaft may be coupled to an electric motor via at least two linkages. A first linkage may be a gear assembly rotatably coupling the shaft to the electric motor to achieve rotation of the shaft. A second linkage may define a prismatic end joint abutting an end of the shaft to cause translation or reciprocal translation of the shaft within the handpiece and/or the capsule.

As referred to herein, reference to an electric motor may include reference to an assembly or arrangement of electric motors configured to achieve a desired motion of the shaft.

In some embodiments, the shaft may actuate a mixing member for achieving mixing. In some embodiments, the shaft may also actuate a puncturing member for puncturing a diaphragm isolating the powder from the liquid, and vice versa. In some cases, the mixing member and the puncturing member may be the same member or in unitary construction. In some embodiments, the shaft may actuate an injector for injecting liquid into the chamber. In some embodiments, the puncturing member may be the same as the injector. For example, puncturing may be achieved by actuation of the needle (or injector needle) suitable for puncturing a diaphragm, e.g. by translation of the needle. The needle may be fluidly connected to a liquid-carrying barrel having a plunger disposed therein. Actuation of the plunger, to achieve translation of the plunger, by the shaft may cause ejection or emission of liquid from the needle.

In some embodiments, a nozzle of the handheld device may receive the paste from the handpiece to eject the paste therefrom. In some embodiments, the shaft may push the paste into and out of the nozzle to extrude the paste therefrom. In some embodiments, the an end connected to the shaft may be protrude at least partially into the nozzle to the paste out of the tip of the nozzle.

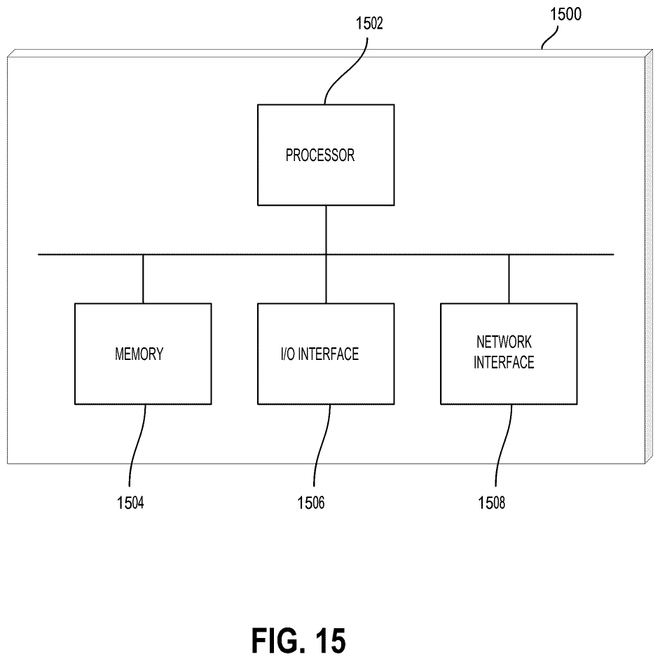

The handheld device(s) may include one or more computing device(s), e.g. including one or more processor(s) and/or as illustrated in FIG. 15, to actuate the various components of the handheld device. For example, computing device(s) may receive data or electrical signals, transmit data or electrical signals to (including to achieve actuation of) various components of the handheld devices to achieve a desired target operation of the handheld device and/or execute one or more methods associated with the handheld device, as described herein.

In various embodiments, a puncturing end, a puncturing member, a central, an electric motor, a plunger, an injector, a mixing member, a mixing end, one or more springs, one or more electromagnets, one or more valves, one or more sensors, and/or one or more power electronic circuits may be actuatable by the one or more computing device(s).

In various embodiments, the handheld device may include one or more (or a plurality) of buttons for actuating the handheld device, and/or for interfacing (e.g. via an input/output interface) with one or more computing device(s) of the handheld device.

Advantageously, combining and integrating components in a handheld device to allow interfacing and sequential or parallel operation of components to generate a stream of dental paste may improve user convenience. Furthermore, such integrating may facilitate provision of specific modes of operation of the handheld device to achieve target paste properties, e.g. consistency and other properties.

FIG. 4B is a schematic cross-sectional view of a handheld device powered by hand, in accordance with the embodiment.

The handheld device may be powered by hand to extrude paste out therefrom. In various embodiments, the handheld device may operate as a caulking gun.

The handheld device may include a crank that is rotatable, e.g. for water injection. In some embodiments, the crank may be rotatable to perform one or more functions of the electric motor. In some embodiments, the device may include a trigger, e.g. to achieve dispensing. The trigger may provide leveraging for pushing paste out of the handheld device.

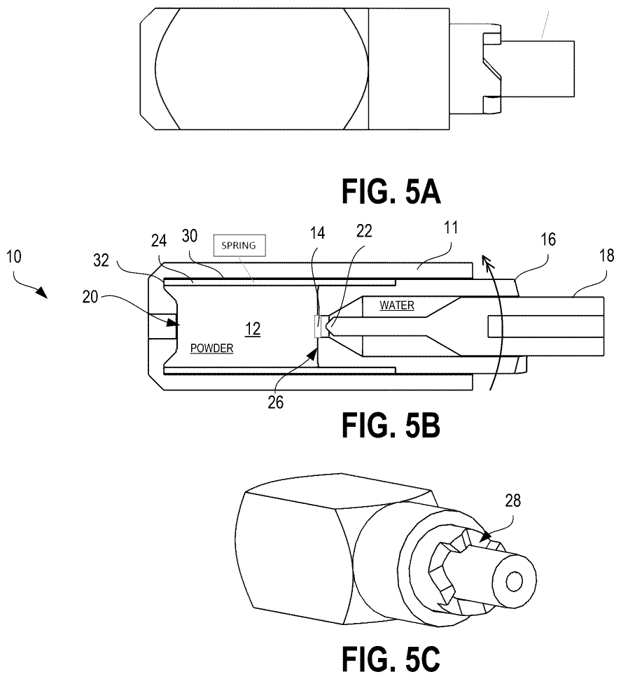

FIG. 5A is a side elevation view of a handheld device, in accordance with an embodiment. FIG. 5B is a cross-sectional view of the handheld device of FIG. 5A, in accordance with an embodiment. Double-headed may generally be used to denote a direction of motion (rotation and/or translation).

FIG. 5C is a perspective view of the handheld device of FIG. 5A, in accordance with an embodiment.

The handheld device 10 includes a housing 11 defining therein a chamber 12 suitable for receiving the powder and the liquid for mixing the powder and the liquid to form the cementitious paste. In various embodiments, the chamber may be tubular. For example, the housing 11 may form part of a handpiece.

The chamber may define an extrusion end 20 for drawing out the cementitious paste after it is formed in the chamber. In various embodiments, a nozzle may be coupled to the extrusion end of the chamber to receive the paste to form a stream of the paste.

A puncturing end 22 may be drivable into the chamber to puncture a diaphragm 14 separating the powder and the liquid in the chamber. The puncturing end may be an end of a puncturing member 18 disposed at least partially coaxially with the chamber.

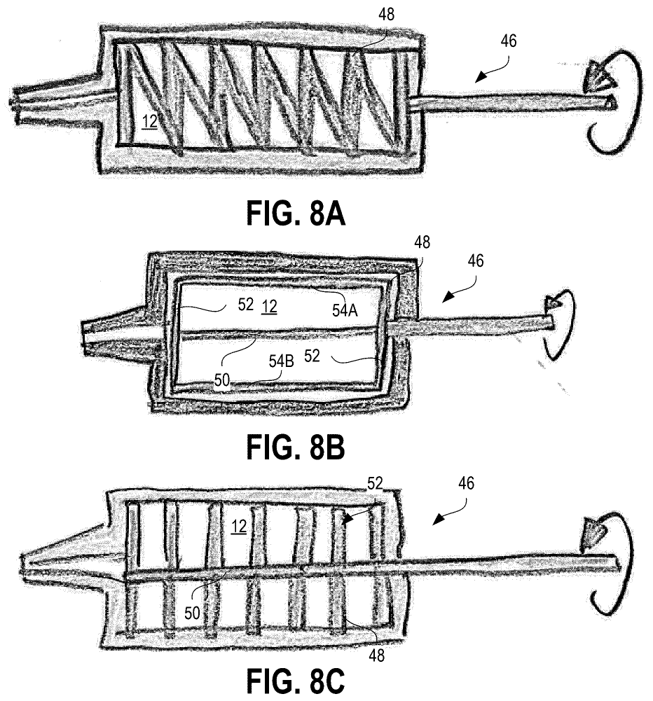

In various embodiments, a spring 24, e.g. a coil or helical spring, may extend into the chamber and may be engaged with the chamber. The spring 24 may be rotatably drivable for mixing the powder and the liquid in the chamber. In general, the spring 24 may be any other suitable mixing end, e.g. as described in relation FIGS. 8A-8C, forming part of a mixing member.

In various embodiments, the coil spring may be at least partially coaxially arranged within the chamber. In some embodiments, the coil spring may abut an inner wall 30 of the chamber. In some embodiments, the coil spring may be compressed, e.g. longitudinally along or coaxially with the chamber, to frictionally engage with an end 32 of the chamber.

In some embodiments, a coupler 16 may be arranged at least partially coaxially with the chamber. The coupler may extend at least partially coaxially at least partially within the chamber and may be suitable for coupling to a driver. The coupler may push against the coil spring to compress the coil spring. The coupler may be frictionally engaged with the coil spring to allow rotation of the coil spring by rotation of the coupler.

In some embodiments, the diaphragm may seal an opening at the end of the coupler. The puncturing member may be disposed inside the coupler. The puncturing member may be movable within the coupler to be releasable into the chamber via an end of the coupler disposed in the chamber to puncture the diaphragm to release the puncturing member into the chamber. In some embodiments, the coupler may be rotatable relative to the puncturing member at least partially coaxially with the chamber.

In some embodiments, the chamber may include an opening for receiving the liquid. In some embodiments, the opening may be coaxial with the mixing member and a puncturing member defining the puncturing end.

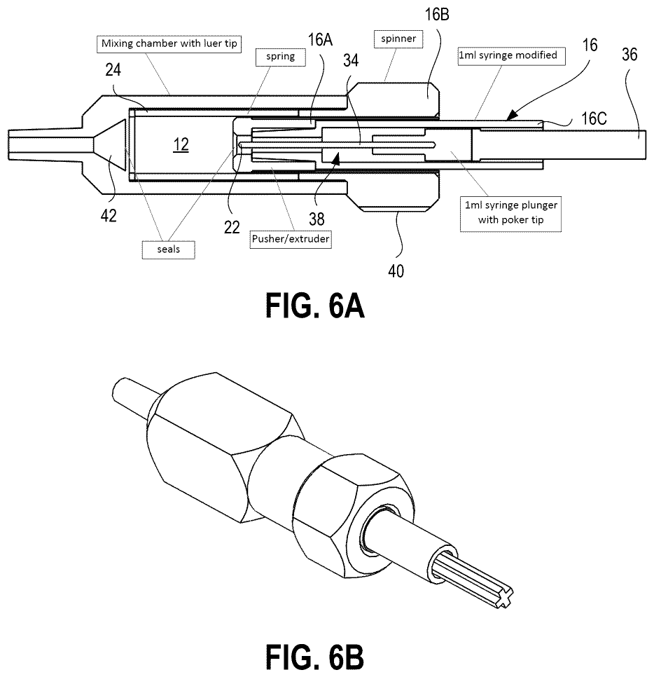

In some embodiments, the coupler defines a front face 26 disposed within the chamber and a rear face 28 accessible from outside the chamber. In some embodiments, the front face forms an end of the chamber. The front face, or a portion of the coupler adjacent to the front face, may be frictionally engaged with spring. In some embodiments, the rear face defines a coupling surface for coupling with a driver, e.g. an electric motor driving a shaft. The spring may be rotatably drivable by the driver via the rear face for mixing the powder and the liquid in the chamber. FIG. 6A is a schematic cross-sectional view of a handheld device, in accordance with the embodiment.

FIG. 6B is a perspective view of the device of FIG. 6A, in accordance with the embodiment.

In FIGS. 6A-6B, the coupler may be a multi-part coupler, including a first part 16A, second part 16B, and third part 16C.

In some embodiments, the puncturing end is an end of a needle 34 coupled to a barrel 38 containing the liquid. In some embodiments, a plunger 36 may be disposed within the barrel 38 and is actuatable to inject the liquid from the barrel into the chamber to cause mixing of the liquid and the powder to form the paste.

In some embodiments, the coupler may be suitable for coupling with the driver by circumferential engagement of a radially outer surface 40 of the coupler with a radially inner surface of the driver.

In reference to FIGS. 6A-6B, the spring may be cause mixing but may not be compressed when dispensing, e.g. only the syringe/plunger may be depressed when dispensing. The needle tip on the end of the plunger may first puncture the water seal (the first seal or diaphragm). A second seal or diaphragm may seal the chamber. The second seal may be spaced apart from the first seal. After mixing, a user may push the plunger in to puncture the second diaphragm to release the paste into a nozzle 42.

The first and second diaphragm and the puncturing end may be arranged around a common axis such that the second diaphragm may be punctured only after puncturing the first diaphragm.

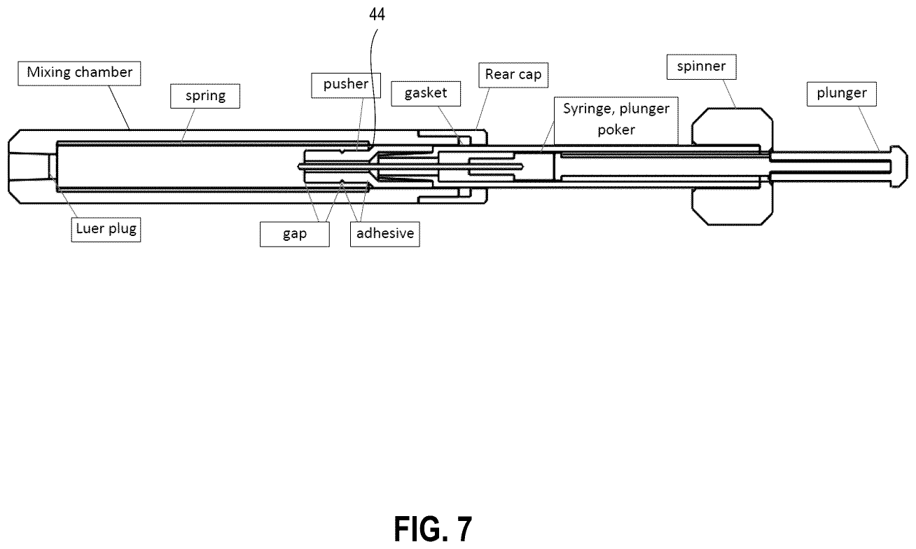

FIG. 7 is a schematic cross-sectional view of a handheld device, in accordance with the embodiment.

In reference to FIG. 7, a smaller diameter and longer spring may be used to maintain mixing volume. A gasket may be added to prevent material from leaking out the back of the unit. The spring may be bonded to the pusher partially in the region marked ‘adhesive’. The region marked ‘gap’ may have no adhesive so that when the plunger is depressed to dispense mixed material, the spring may compress/stack on the outside of the plunger. This means that the face of the plunger may contact the end of the mixing chamber to dispense ‘all’ of the mixed material. A Luer™ plug may extend into the mixing chamber to mitigate loose powder getting stuck in the exit path orifice. The plug may protrude inside and may act as the mixing chamber seal.

In some embodiments, an outer end 44 of the coupler may radially tapered and defines a gasket abutting an end of the chamber to sealably close the end of the chamber.

FIG. 8A is schematic cross-sectional view of a mixing member 46 disposed in a chamber, in accordance with an embodiment.

FIG. 8B is schematic cross-sectional view of a mixing member 46 disposed in a chamber, in accordance with an embodiment.

FIG. 8C is schematic cross-sectional view of a mixing member 46 disposed in a chamber, in accordance with an embodiment.

In reference to FIGS. 8A-8C, various mixing members 46 are shown with corresponding mixing ends 48. The mixing members may extend into the chamber and may be rotatably drivable for mixing the powder and the liquid in the chamber. In various embodiments, the mixing member includes or may be connected to one or more couplers 16. In some embodiments, the front face of the coupler may be frictionally engaged with the mixing end.

In various embodiments, the mixing ends may extend at least partially coaxially along, and within, the chamber for rotation within the chamber. In some embodiments, the mixing member includes the coupler 16 connected to the mixing end to allow rotation of the mixing end by rotation of the coupler. In various embodiments, the coupler may extend at least partially coaxially at least partially within the chamber and may be suitable for coupling to a driver, e.g. an electric motor.

In FIG. 8A, the mixing end is a coil or helical spring. The coil or helical spring may agitate the powder and fluid by generating turbulent flow within the chamber. For example, rotation of the coil or helical spring may generate a vortical flow within the chamber to enhance mixing. The coil spring may be pressed against an inner surface of the chamber to mitigate build up of materials on the inner walls of the chamber.

In reference to FIGS. 8B-8C and other some embodiments, the mixing member may comprise a rotatable central shaft 50 disposed in the chamber and a plurality of members 52 extending radially from the central shaft 50 to provide agitation to mix the powder and the liquid. In reference to FIG. 8B, the mixing end 48 may in some embodiments, a first member 54A of the mixing member extends parallel to the central shaft and is radially spaced apart from the central shaft, and a second member 54B of the mixing member opposed the first member extends parallel to the central shaft and is spaced apart from the central shaft. The first and second members 54A, 54B may be rotatable to cause whisking.

In reference to FIG. 8C, the mixing end 48 may comprise a plurality of radially extending elements. For example, in some embodiments, the mixing end 48 may be a brush comprising a plurality of thin mixing elements or vanes.

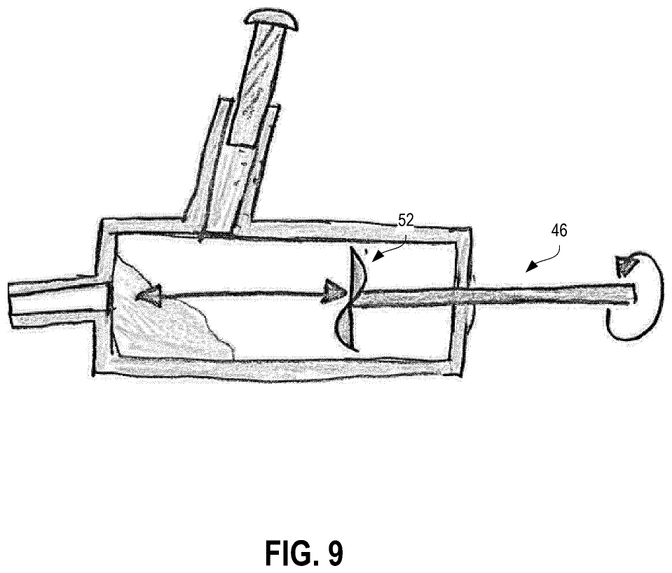

FIG. 9 is schematic cross-sectional view of a mixing member disposed in a chamber having an opening for liquid, in accordance with an embodiment.

In some embodiments, the opening opens at least partially lateral to a central axis of the chamber to receive liquid therefrom into the chamber. A plunger may push the liquid into the chamber. The mixing end may define one or more vanes (e.g. a propeller). The mixing member may combine rotational motion and translational motion in the chamber.

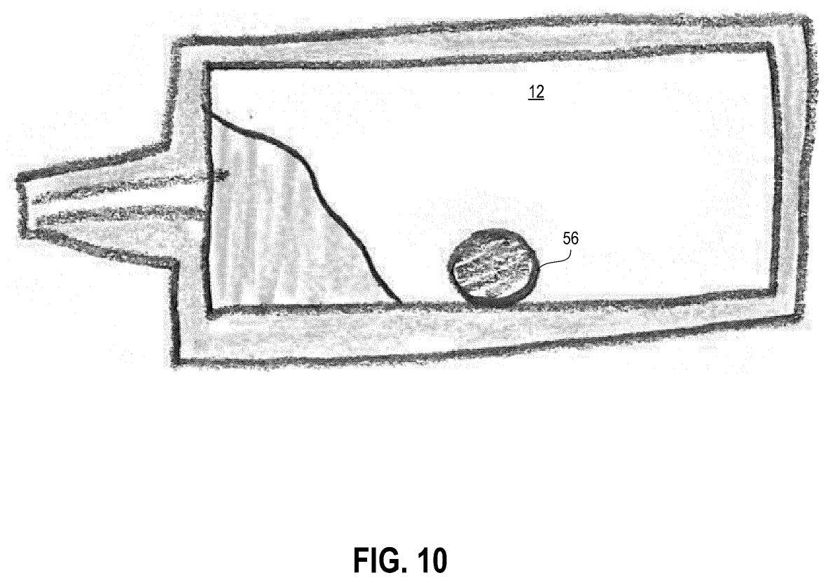

FIG. 10 is schematic cross-sectional view of a mass disposed in a chamber for mixing, in accordance with an embodiment.

In some embodiments, the chamber includes an agitating ball for mixing the powder and the liquid when the chamber is shaken.

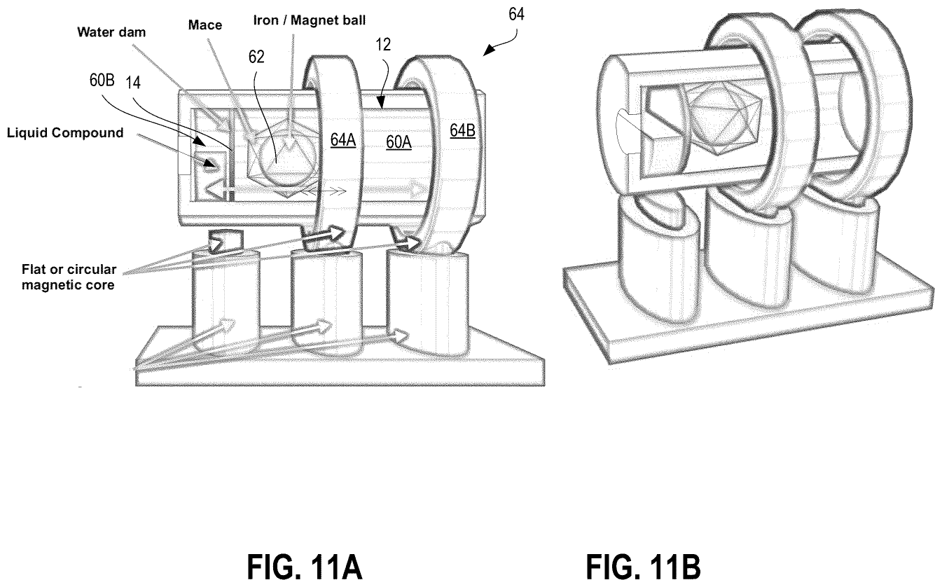

FIG. 11A is a partial sectional view of a handheld device disposed inside a magnetic actuator, in accordance with an embodiment.

FIG. 11B is a perspective partial sectional view of the handheld device of FIG. 11A, in accordance with an embodiment.

The handheld device may includes a chamber 12 defining an extrusion end for drawing out the cementitious paste.

The diaphragm 14 may be disposed in the chamber to separate the chamber into a first volume 60A containing the powder and a second volume 60B containing the liquid.

The diaphragm may be suitable for isolating the powder and the liquid.

A magnetic mass 62 may be disposed inside the chamber to provide agitation. In some embodiments, the magnetic mass may be disposed inside the first volume before puncturing the chamber. In various embodiments, magnetic mass may be substantially freely moving within the chamber, subject to forces arising from electromagnetic field. In some embodiments, the magnetic mass may be ensconced within a cage or a mace.

The magnetic mass may be magnetically actuatable by one or more electromagnets 64. The electromagnets may generate a magnetic field which may exert a force on the magnetic mass, causing it to move. In some embodiments, the one or more electromagnets may be configured to generate a magnetic field inside the chamber to cause reciprocating motion of the magnetic mass. Such movement within the chamber, if sufficiently fast, may puncture the diaphragm to intermingle the powder and the liquid. Following intermingling, the magnetic mass may continue to be moved, e.g. rapidly and/or reciprocally moved, within the chamber to facilitate and enhance mixing of the powder and the liquid.

In some embodiments, the magnetic mass may have a surface suitable for abrasively puncturing the diaphragm and mixing the powder and the liquid, e.g. the magnetic mass may have a roughened surface or a surface with features protruding therefrom.

In some embodiments, the one or more electromagnets may include a plurality of coils 64 at least partially surrounding (externally) the chamber to generate the magnetic field inside the chamber. In some embodiments, a first current passes through a first coil and a second current passes through a second coil. The first and second currents may be adapted to generate a time-varying magnetic field that causes substantially continuous agitation of the powder and the liquid inside the chamber by magnetically actuating the magnetic mass by the time-varying magnetic field.

In various embodiments, the one or more electromagnets include a first electromagnet 64A and a second electromagnet 64B. The first electromagnet and the second electromagnet may be at least partially circular, or fully circular, and may be spaced apart from each other. The chamber may be tubular and may extend from within the first electromagnet to within the second electromagnet such that the magnetic mass may be magnetically actuated by the one or more electromagnets to move between the first electromagnet and the second electromagnet within the chamber.

In some embodiments, the magnetic mass may be made to levitate within the chamber. In some embodiments, the magnetic mass may be made to move within the chamber without hitting the sides of the chamber.

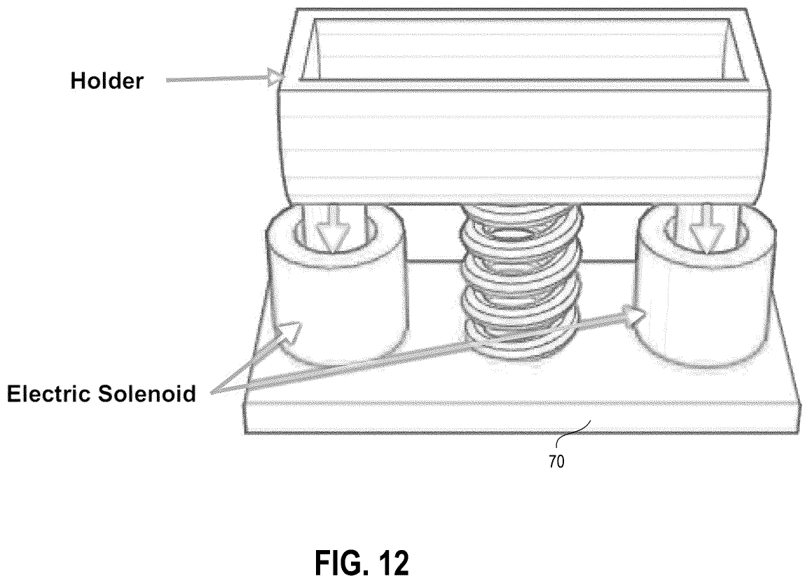

FIG. 12 is a perspective view of a device for mixing liquid and powder, in accordance with an embodiment.

The handheld device may be configured to shake to mixing powder and liquid to form a cementitious paste. The handheld device also includes a holder for receiving a capsule containing the powder and the liquid. One or more linear actuators, e.g. electric solenoids, may couple a base 70 to the holder. The linear actuators may be actuatable to shake the holder to mix the powder and the liquid inside the capsule.

In some embodiments, the chamber shown in FIG. 10 may be used in conjunction with the device of FIG. 12. Shaking may be effective at cause agitation by the mass (or ball) inside the chamber.

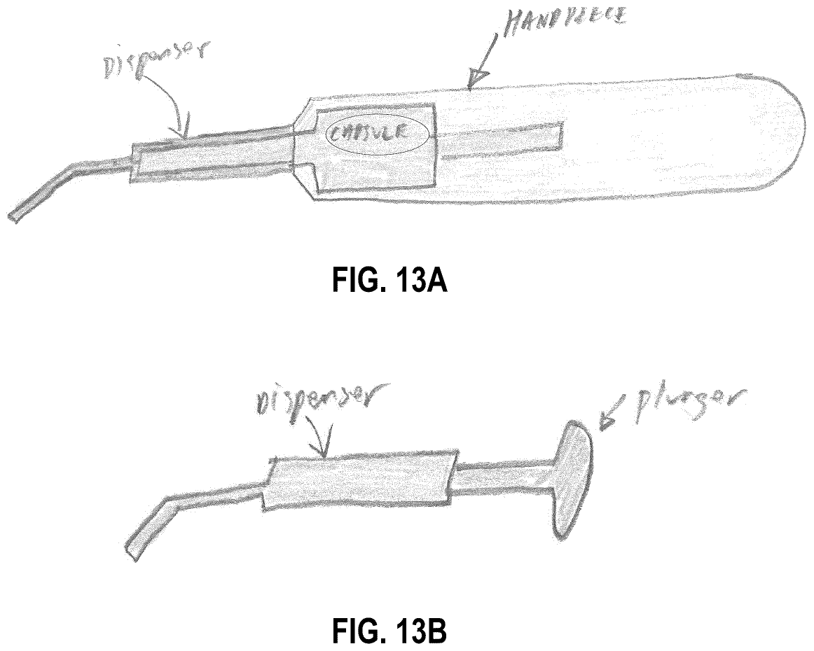

FIG. 13A is a schematic side-view of a device for generating a paste using liquid and powder during a first step, in accordance with an embodiment.

FIG. 13B is a schematic side view of a device for generate the paste during a second step, in accordance with an embodiment.

During the first step, the material from the capsule may be mixed in the chamber. Thereafter, the paste (material) is transferred from the chamber to dispenser. The dispenser is then removed from the handpiece. Thereafter, during a second step, the plunger is inserted into the dispenser to allow application of the paste.

Advantageously, specialized nozzle may be utilized to prevent separation of liquid from the paste. For example, a plurality of nozzles may be sequentially arranged to cause gradual extrusion of the paste down to a desired extrusion diameter (e.g. 18 gauge) from the handheld device in order to prevent separation of liquid from the paste.

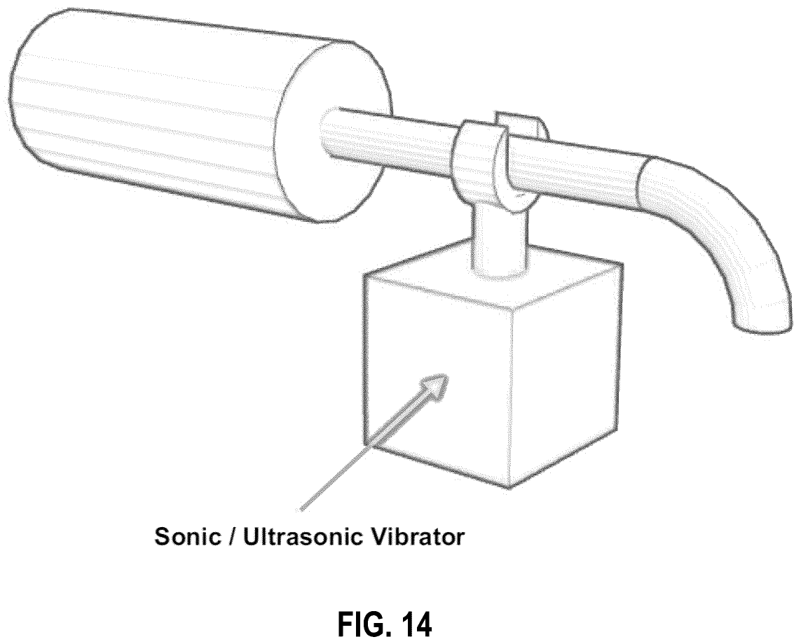

FIG. 14 is a perspective view of a nozzle couple to a device and a vibrator, in accordance with an embodiment.

In some embodiments, the nozzle is coupled to a vibrator to vibrate the stream. In some embodiments, the vibrator is at least one of a sonic or ultrasonic vibrator. The vibrator may be captively engaged with an outer end of the nozzle tip.

In various embodiments, the tip where the materials are injected from may be adapted for surgical use. In some embodiments, the tip may be adapted to be complementary to a surgical (ultrasonic tip). Surgical ultrasonic tips may have one or more shapes, e.g. a surgical ultrasonic tup may have a shape that is based on and/or depends on the location of the tooth. For example, the tip may mimic the shape of the surgical ultrasonic tips that are used to make the retro-preparation cavities in apicoectomy surgeries such that when a surgeon makes a cavity at the end of the root with a specific shaped surgical ultrasonic tip, the handheld device may be implemented and used with a tip with a shape similar to or complementary to the surgical ultrasonic tip to inject the material inside the cavity.

In various embodiments, the injector tip may be straight or it may have one or two bends. In various embodiments, the injector tip may be flexible or rigid.

FIG. 15 illustrates a block diagram of a computing device 1200, in accordance with an embodiment of the present application.

In some embodiments, as an example, the handheld device(s), system configured to work handheld device(s), actuators, electric motors, electromagnets, solenoids, and/or systems and subsystems may be at least partially implemented using the example computing device 1200 of FIG. 15.

The computing device 1200 includes at least one processor 1202, memory 1204, at least one I/O interface 1206, and at least one network communication interface 1208.

The processor 1202 may be a microprocessor or microcontroller, a digital signal processing (DSP) processor, an integrated circuit, a field programmable gate array (FPGA), a reconfigurable processor, a programmable read-only memory (PROM), or combinations thereof.

The memory 1204 may include a computer memory that is located either internally or externally such as, for example, random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM).

The I/O interface 1206 may enable the computing device 1200 to interconnect with one or more input devices, such as a keyboard, mouse, camera, touch screen and a microphone, or with one or more output devices such as a display screen and a speaker.

The networking interface 1208 may be configured to receive and transmit data sets representative of the machine learning models, for example, to a target data storage or data structures. The target data storage or data structure may, in some embodiments, reside on a computing device or system such as a mobile device.

The term “connected” or “coupled to” may include both direct coupling (in which two elements that are coupled to each other contact each other) and indirect coupling (in which at least one additional element is located between the two elements).

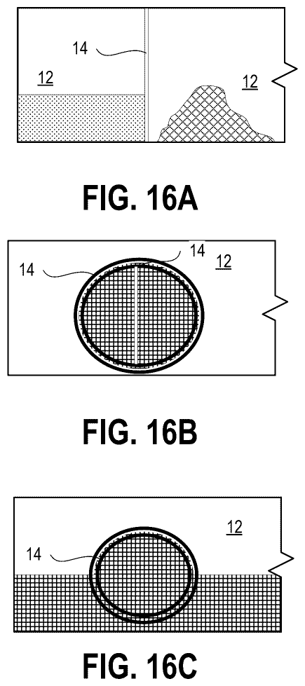

FIG. 16A is a schematic sectional view of a chamber with a diaphragm separating the powder and the liquid, in accordance with an embodiment.

In some embodiments, the diaphragm is coupled to the chamber to separate the chamber in two separate portions or volumes. The puncturing end may puncture the diaphragm to cause intermingling of powder and water.

FIG. 16B is a schematic sectional view of a chamber with a single capsule having two compartments, in accordance with an embodiment.

FIG. 16C is a schematic sectional view of a chamber with a capsule disposed therein, in accordance with an embodiment.

In reference to FIGS. 16B-16C and in other such embodiments, the diaphragm may be a membrane of a capsule. In some embodiments, the membrane is an outer covering of the capsule. In various embodiments, the capsule may contain liquid and/or powder. In some embodiments, the capsule may define two isolated volumes or compartments, as shown in FIG. 16B, one each for the liquid and the powder. The puncturing end may puncture the outer membrane of the capsule, and possibly also the central membrane, e.g. if the compartments have separate outer membranes, to cause intermingling of the powder and water.

In some embodiments, as shown in FIG. 16C, a capsule may provided with a first material (liquid or powder) and a second material (powder or liquid, respectively) may be disposed in the chamber external to the capsule. The puncturing end may puncture the outer membrane of the capsule to cause intermingling of powder and liquid.

In some embodiments, two separate capsules may be provided. A first capsule may contain liquid and a second capsule may contain powder.

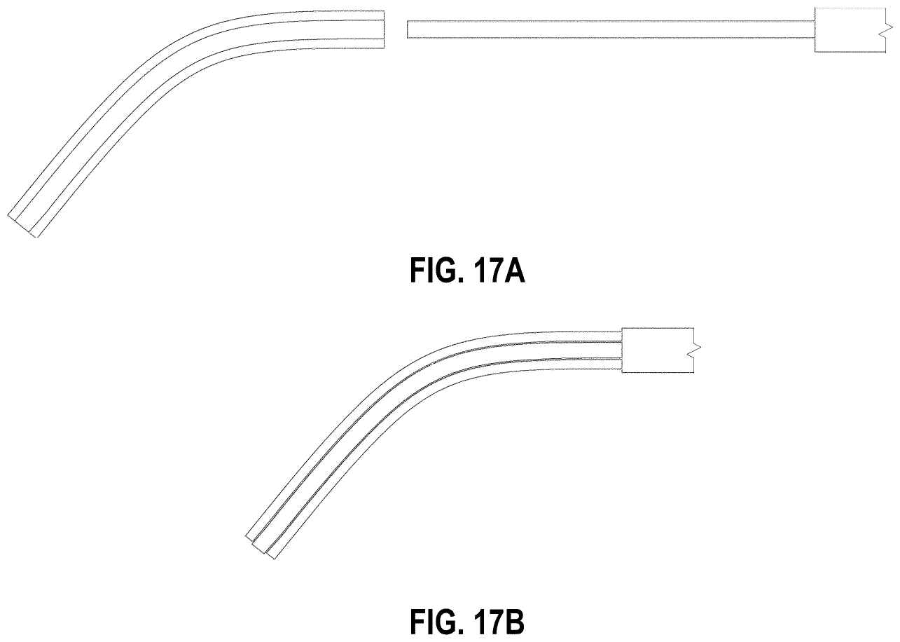

FIG. 17A is a cross-sectional view of a nozzle and a forward portion of a plunger, in accordance with an embodiment.

FIG. 17B is a cross-sectional view of the nozzle and the forward portion of the plunger of FIG. 17A with the plunger engaged with the nozzle, in accordance with an embodiment.

The plunger, or a portion thereof at a forward end of the plunger, e.g. the forward portion of the plunger, may be flexible. In various embodiments, the plunger may be constructed of a flexible polymer. Advantageously, the flexible plunger may facilitate dispensing of the contents of the dispensing tip, e.g. in some cases the entire contents of the dispensing tip may be dispensed, and may be particularly advantageous when the dispensing tip is curved, as shown in FIGS. 17A-17B.

In various embodiments, the forward portion of the plunger may be part of the mixing member and/or a puncturing member.

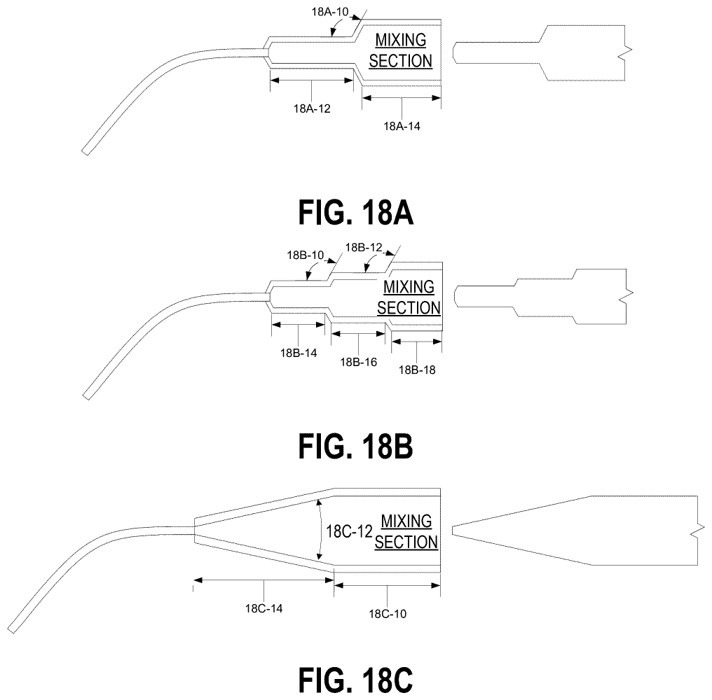

FIG. 18A is a cross-sectional view of a nozzle and a forward portion of a plunger, in accordance with another embodiment.

The nozzle and plunger of FIG. 18A may be part of a 3-stage dispensing system. A first stage may have a length 18A-14, and a second stage may have a length 18A-12. A third (and final) stage may be a curved tip portion or a portion that is at or narrows to a desired width, e.g. 18 gauge.

In some embodiments, the length 18A-14 may be greater than the length 18A-12. The diameter (e.g. cross-section normal to the direction in which the paste flows) of the first stage may be larger than the diameter of the second stage. The diameter of the second stage may be larger the diameter of the dispensing tip. The nozzle may be tapered at a constant or variable angle between the first stage and the second stage. For example, such a taper may be constant and defined by an angle 18A-10 between 90° and 180°

Advantageously, in some cases, such a staged or sequentially tapered device may allow efficient and thorough extrusion of paste from the dispensing tip by pushing of a plunger complementary to the nozzle, and engaged with the nozzle, into the nozzle.

FIG. 18B is a cross-sectional view of a nozzle and a forward portion of a plunger, in accordance with yet another embodiment.

The nozzle and plunger of FIG. 18B may be part of a 4-stage dispensing system. A first stage may have a length 18B-18, a second stage may have a length 18A-16, and a third stage may have a length of 18A-14. A fourth (and final) stage may be a curved tip portion or a portion that is at or narrows to a desired width, e.g. 18 gauge. The nozzle may be tapered at a constant or variable angle between the first stage and the second stage, and the second stage and the third stage. For example, such a taper may be constant and defined by an angle 18A-10 between 90° and 180° and angle 18A-10 between 90° and 180°. For example, the angle 18A-10 and 18A-12 may be substantially equal.

The diameter of the first stage may be larger than the diameter of the second stage. The diameter of the second stage may be larger than a diameter of the third stage. The diameter of the third stage may be larger than the diameter of the dispensing tip.

FIG. 18C is a cross-sectional view of a nozzle and a forward portion of a plunger, in accordance with a yet further embodiment.

The nozzle and plunger of FIG. 18C may be part of a substantially continuously tapering dispensing system. A first stage may have a length 18C-10, and a second stage may have a length 18C-14 (projected length on to a direction parallel to the walls of the first stage). The taper may be defined by an angle 18C-12 between 0° and 180°.

In reference to FIGS. 18A-18C, the plunger may pushed against material in the nozzles to cause mixing and gradual extrusion to mitigate separate of liquid from the material.

It is understood that in various embodiments, the nozzles and complementary plungers may comprise a plurality of stepped sections or stages. For example, FIG. 18A shows a system with three stepped sections, and FIG. 18B shows four such section. In various embodiments, additional sections may be added.

It is understood that in various embodiments, the nozzles and complementary plungers may comprise one or more tapered sections and/or one or more stepped sections.

As can be understood, the examples described above and illustrated are intended to be exemplary only; the embodiments described in this document provide non-limiting examples of possible implementations of the present technology. Upon review of the present disclosure, a person of ordinary skill in the art will recognize that changes may be made to the embodiments described herein without departing from the scope of the present technology. Modifications could be implemented by a person of ordinary skill in the art in view of the present disclosure, which modifications would be within the scope of the present technology.

As one of ordinary skill in the art will readily appreciate from the disclosure, processes, machines, manufacture, compositions of matter, means, methods, or steps, presently existing or later to be developed, that perform substantially the same function or achieve substantially the same result as the corresponding embodiments described herein may be utilized. Accordingly, the embodiments are intended to include within their scope such processes, machines, manufacture, compositions of matter, means, methods, or steps.

What is claimed is:

1. An improved MTA powder composition that produces a filling material paste when mixed with water for use in general dentistry or endodontics to replace natural tooth material, the composition comprising:
a. Portland cement;
b. extrinsic calcium sulfate as a setting enhancer; and
c. a radio pacifier material.

2. The composition of claim 1 comprising:
a. 60-70% by weight of the Portland cement;
b. 10-15% by weight of the extrinsic calcium sulfate; and
c. 15-20% by weight of the radio pacifier material.

3. The composition of claim 1, wherein the Portland cement component of the composition comprises:
a. 45-80% by weight of the Portland cement of Tri Calcium Silicate;
b. 10-25% by weight of the Portland cement of intrinsic Calcium Sulfate;
c. 5-15% by weight of the Portland cement of Hibschite (Al₂Ca₂(SiO₄)2.16 (OH)3.36); and
d. 5-15% by weight of the Portland cement of Calcium Aluminum Oxide Carbonate (Ca₄Al₂O₆CO₃·11H₂O).

4. The composition of claim 3 comprising:
a. 55-75% by weight of the Portland cement;
b. 15-25% by weight of the extrinsic calcium sulfate; and
c. 10-20% by weight of the radio pacifier material consisting of barium titanate.

5. The composition of claim 1 comprising:
a. 65-85% by weight of the Portland cement;
b. 0-10% by weight of the extrinsic calcium sulfate; and
c. 15-25% by weight of the radio pacifier material consisting of barium titanate.

6. The composition of any one of claims 1-3, wherein the radio pacifier material is selected from one or more of barium titanate, bismuth oxide, and zirconium oxide.

7. The composition of claim 6 wherein the radio pacifier material is barium titanate.

8. A kit to produce a cementitious paste filler for use in general dentistry or endodontics to replace natural tooth material, the kit comprising:
a. a first capsule containing an amount of the powder composition as claimed in any one of claims 1-7;
b. a second capsule containing a volume of water; and
c. instructions to mix the volume of water with the amount of the powder composition to form the cementitious paste filler.

9. The kit as claimed in claim 8 wherein the ratio of the volume weight of the powder composition in the first capsule to the weight of the water in the second capsule is in the range of 3:1-3.

10. A method of filling a natural, pathogenic, or iatrogenic, tooth cavity, comprising the steps of:
a. identifying the cavity of the tooth to be filled;
b. mixing a weight of the powder composition as claimed in any one of claims 1-7 with a volume of water to produce a filling material paste;
c. introducing the filling material paste into the tooth cavity; and
d. allowing the filling material paste to harden for a duration of less than 10 minutes.

11. The method of claim 10 wherein the ratio of the weight of the powder composition to the volume of the water is in the range of 3:1.

12. The method of claim 10 wherein the ratio of the weight of the powder composition to the volume of the water is in the range of 3:2.

13. A handheld device for forming a cementitious paste from a powder and a liquid, comprising:
a chamber suitable for receiving the powder and the liquid for mixing the powder and the liquid to form the cementitious paste, the chamber defining an extrusion end for drawing out the cementitious paste;
a puncturing end drivable into the chamber to puncture a diaphragm separating the powder and the liquid in the chamber; and
a mixing member extending into the chamber and being rotatably drivable for mixing the powder and the liquid in the chamber.

14. A method of generating a stream of cementitious paste using powder and liquid, comprising:
puncturing a diaphragm in a chamber to intermingle the powder and the liquid;
rotatably driving a mixing member in the chamber to mix the powder and the liquid to form a cementitious paste; and
extruding the cementitious paste through an opening in the chamber to form the stream of the cementitious paste.

15. A handheld device for forming a cementitious paste from a powder and a liquid, comprising:
a chamber defining an extrusion end for drawing out the cementitious paste;
a diaphragm disposed in the chamber to separate the chamber into a first volume containing the powder and a second volume containing the liquid, the diaphragm suitable for isolating the powder and the liquid; and
a magnetic mass disposed inside the chamber to provide agitation, the magnetic mass being actuatable by one or more electromagnets to move within the chamber to puncture the diaphragm and to mix the powder and the liquid.

16. A method of generating a stream of cementitious paste using powder and liquid, comprising:
moving a magnetic mass in a chamber by magnetic actuation to puncture a diaphragm in the chamber to intermingle the powder and the liquid;
after intermingling of the powder and the liquid, moving the magnetic mass in the chamber by magnetic actuation to mix the powder and the liquid to form a cementitious paste; and
extruding the cementitious paste through an opening in the chamber to form the stream of cementitious paste.

17. A handheld device for mixing powder and liquid to form a cementitious paste, comprising:
a holder for receiving a capsule containing the powder and the liquid;
a base; and
one or more linear actuators coupling the base to the holder, the linear actuators being actuatable to shake the holder to mix the powder and the liquid inside the capsule.

18. A method of mixing powder and liquid, comprising
receiving a capsule containing the powder and the liquid in a holder, and
actuating a plurality of linear actuators coupled to the holder to shake the holder to mix the powder and the liquid inside the capsule.
